(19) United States
(12) Patent Application Publication
(10) Pub. No.: US 2021/0047423 A1
(43) Pub. Date: Feb. 18, 2021

(54) THERAPEUTIC IMMUNE CELLS WITH IMPROVED FUNCTION AND METHODS FOR MAKING THE SAME

(71) Applicant: Janssen Biotech, Inc., Horsham, PA (US)

(72) Inventors: Shan HE, Blue Bell, PA (US); Michael KALOS, Wayne, PA (US)

(21) Appl. No.: 16/992,196

(22) Filed: Aug. 13, 2020

Related U.S. Application Data
(60) Provisional application No. 62/887,800, filed on Aug. 16, 2019.

(51) Int. Cl. C07K 16/28; C07K 14/47; C12N 15/86; A61K 35/17

(52) U.S. Cl. CPC C07K 16/2878; C07K 14/4702; C12N 2740/15043; A61K 35/17; C12N 15/86

(57) ABSTRACT

The present disclosure provides for immune cells with improved function and properties. Immune cells that overexpress, or ectopically express, autophagy modulators are provided. Increased expression of autophagy modulators can improve the function of a T cell or an NK cell expressing chimeric antigen receptors (CARs). For example, CAR-T cells that ectopically express ATG5 or ATG7 exhibit improved stimulation in response to antigen.

### CROSS-REFERENCE TO RELATED APPLICATIONS

This application claims the benefit of U.S. Provisional Application Ser. No. 62/887,800, filed 16 Aug. 2019. The entire content of the aforementioned application is incorporated herein by reference in its entirety.

### SEQUENCE LISTING

The instant application contains a Sequence Listing which has been submitted electronically in ASCII format and is hereby incorporated by reference in its entirety. Said ASCII copy, created on Jul. 30, 2020, is named JBI6140USNP1_SL.txt and is 22,954 bytes in size.

### TECHNICAL FIELD

The invention relates to methods for improving functional attributes of immune cells expressing chimeric antigen receptors (CARs). Modulation of the function of the autophagy pathway in a CAR-T cell can be undertaken, for example, by ectopically expressing one or more autophagy pathway modulators (e.g., ATG5 or ATG7) in the CAR-T cell so as to improve the function and survival of the CAR-T cell. The function and survival of tumor infiltrating lymphocytes can also be improved by ectopically expressing autophagy pathway genes in these cells.

### BACKGROUND

Adoptive cell therapy with gene-engineered lymphocytes has shown considerable promise, particularly in hematologic malignancies. However, there is a compelling medical and practical need for cell therapy products that are more potent, safer, and more effective. One particular area of interest is generation of cell products with enhanced proliferative capacity that also retain a more “central memory” and less “exhausted” phenotype. Ideally such products would retain potent anti-tumor activity, expand robustly in the patient, are able to be dosed in lower numbers, and manifest less cytokine release syndrome. Multiple strategies have and continue to be pursued to generate cell therapy products with these properties, including culture and selection conditions as well as gene engineering (e.g., tet-2).

### SUMMARY

Provided herein are therapeutic immune cell (e.g., T cell, NK cell, and B cell) compositions, and methods for making such compositions, that address the above needs for potency, safety and effectiveness. Ectopic expression of autophagy pathway related genes (e.g., ATG5 or ATG7) in CAR T cells, for example, can improve function of these T cells. Without wishing to be bound by theory, modulation of autophagy pathway related genes might drive immune cells toward a more functionally desired metabolic state, leading to enhanced cellular persistence. Conversely, decreases in cellular autophagy might impair the ability of lymphocytes to sustain themselves and to function.

Described herein is the use of lentiviral vector technology to induce ectopic expression of autophagy pathway related genes to adapt the immunometabolism of lymphocyte in order to promote more potent, effective, and safer cell therapy products. The data in this disclosure show that ectopic expression of autophagy pathway genes ATG5 or ATG7 in lymphocytes provides for superior engineered cell therapy products that show enhanced ability to expand upon repetitive exposure to target cells, and superior functional properties as measured by enhanced cytokine and cytotoxicity in response to target cells.

In one aspect is provided an immune cell expressing a first vector comprising a nucleotide sequence encoding a chimeric antigen receptor (CAR) and a second vector comprising a nucleotide sequence encoding an autophagy modulator.

In another aspect is provided an immune cell expressing a vector comprising a first nucleotide sequence encoding a chimeric antigen receptor (CAR) and a second nucleotide sequence encoding an autophagy modulator.

In another aspect is provided an immune cell comprising a vector comprising a nucleotide sequence encoding an autophagy modulator. In one embodiment, the immune cell further comprises a CAR.

In various embodiments of the above aspects, the genome of the immune cell comprises one or more additional autophagy modulator genes. In various embodiments of the above aspects, a promoter of an autophagy modulator gene is replaced with a constitutive promoter (e.g., a TRAC promoter, a β-2m promoter, a CMV promoter, or an EF1a promoter). Replacement of the promoter may be undertaken using a gene editing system (e.g., a CRISPR/Cas9 system, a CRISPR/Cpf1 system a zinc finger nuclease system, a TALEN system, or a meganuclease system). In various embodiments of the above aspects, wherein an enhancer sequence of an autophagy modulator gene is replaced with a second enhancer sequence that is effective to increase transcription of the autophagy modulator gene.

In some embodiments of the above aspects, the immune cell is a lymphocyte. In some embodiments, the immune cell is a tumor penetrating lymphocyte. In various embodiments, the immune cell is a T cell, a Natural Killer (NK) cell, or a B cell.

In various embodiments of the above aspects, the CAR comprises an extracellular domain that specifically binds to the B-cell maturation antigen (BCMA), a CD19 antigen, a CD30 antigen, a CD123 antigen, an FLT3 antigen, and kallikrein-2 antigen.

In some embodiments of the above aspects, the autophagy modulator is ATG1, ATG2, ATG3, ATG4, ATG5, ATG6, ATG7, ATG8, ATG8, ATG10, ATG11, ATG12, ATG13, ATG14, ATG15, ATG16, ATG17, ATG18, ATG19, ATG20, ATG21, ATG22, ATG23, ATG24, ATG25, ATG26, ATG27, ATG28, ATG29, ATG30, ATG31, ATG101, LC3, RAB7, VPS15, VPS34, VPS35, LC3I, LC3II, UVRAG, Beclin1, Protor, CAMKKbeta, BCL2, BCL-XL, AKT, ULK1, ULK2, ULK3, ULK4, DapK1, FIP200, TSC1, TSC2, STRAD, AMPK, Redd1, LKB, M025, PTEN, mTOR, Deptor, Rictor, Protor, PRAS40, LST8, Rheb, RAG A, RAG B, RAG C, RAG D, Raptor, PDK1, PI3K, IRS1, Insulin/IGF1 receptor, ERK, Rab40b, p53, DRAM1, NDFIP, MEK, RAF, SIN1, MAP4K3, Plac8, Dominant negative (DN) Rab7, Rab7, Dominant active (DA) Rab7, SLC7A5, or SLC3A2. In certain embodiments, the autophagy modulator is an epigenetic regulator. Examples of epigenetic regulators include, but are not limited to histone methyltransferase (e.g., EZH2 (enhancer of zeste 2 polycomb repressive complex 2 subunit) or G9a), DNA methyltransferase (e.g., DNMT3), and histone deacetylases.

In some embodiments, the autophagy modulator is ATG5. In some embodiments, the ATG5 comprises an amino acid sequence at least 95% identical to the sequence of MTDDKDVLRDVWFGRIPTCFTLYQDEITEREAEPYYLLLPRVSYLTLVTDKVKKHF QKVMRQEDISEIWFEYEGTPLKWHYPIGLLFDLLASSSALPWNITVHFKSFPEKDLL HCPSKDAIEAHFMSCMKEADALKHKSQVINEMQKKDHKQLWMGLQNDRFDQFW AINRKLMEYPAEENGFRYIPFRIYQTTTERPFIQKLFRPVAADGQLHTLGDLLKEVC PSAIDPEDGEKKNQVMIHGIEPMLETPLQWLSEHLSYPDNFLHISIIPQPTD (SEQ ID NO: 1). In some embodiments, the ATG5 comprises an amino acid sequence at least 95% identical to the sequence of MTDDKDVLRDVWFGRIPTCFTLYQDEITEREAEPYYLLLPRVSYLTLVTDKVKKHF QKVMRQEDVSEIWFEYEGTPLKWHYPIGLLFDLLASSSALPWNITVHFKSFPEKDL LHCPSKDAVEAHFMSCMKEADALKHKSQVINEMQKKDHKQLWMGLQNDRFDQF WAINRKLMEYPPEENGFRYIPFRIYQTTTERPFIQKLFRPVAADGQLHTLGDLLREV CPSAVAPEDGEKRSQVMIHIEPMLETPLQWLSEHLSYPDNFLHISIVPQPTD (SEQ ID NO: 2).

In some embodiments, the autophagy modulator is ATG7. In some embodiments, the ATG7 comprises an amino acid sequence at least 95% identical to the sequence of MAAATGDPGLSKLQFAPFSSALDVGFWHELTQKKLNEYRLDEAPKDIKGYYYNG DSAGLPARLTLEFSAFDMSAPTPARCCPAIGTLYNTNTLESFKTADKKLLLEQAAN EIWESIKSGTALENPVLLNKFLLLTFADLKKYHFYYWFCYPALCLPESLPLIQGPVG LDQRFSLKQIEALECAYDNLCQTEGVTALPYFLIKYDENMVLVSLLKHYSDFFQGQ RTKITIGVYDPCNLAQYPGWPLRNFLVLAAHRWSSSFQSVEVVCFRDRTMQGARD VAHSIIFEVKLPEMAFSPDCPKAVGWEKNQKGGMGPRMVNLSECMDPKRLAESSV DLNLKLMCWRLVPTLDLDKVVSVKCLLLGAGTLGCNVARTLMGWGVRHITFVD NAKISYSNPVRQPLYEFEDCLGGGKPKALAAADRLQKIFPGVNARGFNMSIPMPGH PVNFSSVTLEQARRDVEQLEQLIESHDVVFLLMDTRESRWLPAVIAASKRKLVINA ALGFDTFVVMRHGLKKPKQQGAGDLCPNHPVASADLLGSSLFANIPGYKLGCYFC NDVVAPGDSTRDRTLDQQCTVSRPGLAVIAGALAVELMVSVLQHPEGGYAIASSS DDRMNEPPTSLGLVPHQIRGFLSRFDNVLPVSLAFDKCTACSSKVLDQYEREGFNF LAKVFNSSHSFLEDLTGLTLLHQETQAAEIWDMSDDETI (SEQ ID NO: 3). In some embodiments, the ATG7 comprises an amino acid sequence at least 95% identical to the sequence of MGDPGLAKLQFAPFNSALDVGFWHELTQKKLNEYRLDEAPKDIKGYYYNGDSAG LPTRLTLEFSAFDMSASTPAHCCPAMGTLHNTNTLEAFKTADKKLLLEQSANEIW EAIKSGAALENPMLLNKFLLLTFADLKKYHFYYWFCCPALCLPESIPLIRGPVSLDQ RLSPKQIQALEHAYDDLCRAEGVTALPYFLFKYDDDTVLVSLLKHYSDFFQGQRT KITVGVYDPCNLAQYPGWPLRNFLVLAAHRWSGSFQSVEVLCFRDRTMQGARDV THSIIFEVKLPEMAFSPDCPKAVGWEKNQKGGMGPRMVNLSGCMDPKRLAESSV DLNLKLMCWRLVPTLDLDKVVSVKCLLLGAGTLGCNVARTLMGWGVRHVTFV DNAKISYSNPVRQPLYEFEDCLGGGKPKALAAAERLQKIFPGVNARGFNMSIPMP GHPVNFSDVTMEQARRDVEQLEQLIDNHDVIFLLMDTRESRWLPTVIAASKRKLV INAALGFDTFVVMRHGLKKPKQQGAGDLCPSHLVAPADLGSSLFANIPGYKLGCY FCNDVVAPGDSTRDRTLDQQCTVSRPGLAVIAGALAVELMVSVLQHPEGGYAIAS SSDDRMNEPPTSLGLVPHQIRGFLSRFDNVLPVSLAFDKCTACSPKVLDQYEREGF TFLAKVFNSSHSFLEDLTGLTLLHQETQAAEIWDMSDEETV (SEQ ID NO: 4).

In various embodiments of the above aspects, the vector is a lentiviral vector.

In some embodiments of the above aspects, the expression of the autophagy modulator is at least four times the level of expression of the autophagy modulator in a comparable immune cell with normal expression of the autophagy inhibitor. In some embodiments, the cytotoxic activity of the immune cell is not lower than that of a comparable immune cell with normal expression of the autophagy inhibitor. In some embodiments, the immune cell is able to proliferate to a greater extent than a comparable immune cell with normal expression of the autophagy inhibitor. In some embodiments, the immune cell is a T cell and enters a state of T cell exhaustion at a later time than a comparable immune cell with normal expression of the autophagy inhibitor. In some embodiments, the immune cell is a T cell that does not undergo T cell exhaustion.

In another aspect is provided a pharmaceutical composition comprising an effective amount of any of the immune cells described above, and a pharmaceutically acceptable excipient.

In another aspect is provided a method of preparing any of the immune cells described above, the method comprising introducing the vector (e.g., the first and/or second vectors) into the immune cell. In some embodiments, the vector is transduced into the immune cell. In some embodiments, the vector is a viral vector. In some embodiments, the first vector is a viral vector, the second vector is a viral vector, or both the first vector and the second vector are viral vectors. In various embodiments, a gene editing system is used to introduce the vector into the immune cell. The gene editing system may be selected from the group consisting of a CRISPR/Cas9 system, a CRISPR/Cpf1 system a zinc finger nuclease system, a TALEN system, and a meganuclease system. In various embodiments, the vector is integrated into the genome of the immune cell. In certain embodiments, the vector is integrated into a TRAC locus of the genome.

In another aspect is provided a method of treating a disease or condition comprising administering any of the immune cells described above to a subject.

In another aspect is provided a method of treating a subject having cancer, the method comprising: administering a therapeutically effective amount of any of the immune cells described above to a subject in need thereof, whereby the immune cell induces killing of cancer cells in the subject.

In another aspect is provided a method of reducing T cell exhaustion comprising contacting a T cell with an autophagy modulator and/or a vector comprising a nucleotide sequence encoding the autophagy modulator. In a related aspect is provided a method of reducing T cell exhaustion comprising introducing a nucleotide sequence encoding the autophagy modulator into the T cell using a gene editing system (e.g., a CRISPR/Cas9 system, a CRISPR/Cpf1 system a zinc finger nuclease system, a TALEN system, or a meganuclease system).

In another aspect is provided a method of reducing NK cell exhaustion comprising contacting an NK cell with an autophagy modulator and/or a vector comprising a nucleotide sequence encoding the autophagy modulator. In a related aspect is provided a method of reducing NK cell exhaustion comprising introducing a nucleotide sequence encoding the autophagy modulator into the NK cell using a gene editing system (e.g., a CRISPR/Cas9 system, a CRISPR/Cpf1 system a zinc finger nuclease system, a TALEN system, or a meganuclease system).

In another aspect is provided a method of increasing the proliferation of an immune cell comprising contacting the immune cell with an autophagy modulator and/or a vector comprising a nucleotide sequence encoding the autophagy modulator. In a related aspect is provided a method of increasing the proliferation of an immune cell comprising introducing a nucleotide sequence encoding the autophagy modulator into the immune cell using a gene editing system (e.g., a CRISPR/Cas9 system, a CRISPR/Cpf1 system a zinc finger nuclease system, a TALEN system, or a meganuclease system).

In another aspect is provided a method of improving regulation of effector/memory differentiation of an immune cell, the method comprising contacting the immune cell with an autophagy modulator and/or a vector comprising a nucleotide sequence encoding the autophagy modulator. In a related aspect is provided a method of improving regulation of effector/memory differentiation of an immune cell comprising introducing a nucleotide sequence encoding the autophagy modulator into the immune cell using a gene editing system (e.g., a CRISPR/Cas9 system, a CRISPR/Cpf1 system a zinc finger nuclease system, a TALEN system, or a meganuclease system).

In various embodiments of the above methods, the autophagy modulator is ATG1, ATG2, ATG3, ATG4, ATG5, ATG6, ATG7, ATG8, ATG8, ATG10, ATG11, ATG12, ATG13, ATG14, ATG15, ATG16, ATG17, ATG18, ATG19, ATG20, ATG21, ATG22, ATG23, ATG24, ATG25, ATG26, ATG27, ATG28, ATG29, ATG30, ATG31, ATG101, LC3, RAB7, VPS15, VPS34, VPS35, LC3I, LC3II, UVRAG, Beclin1, Protor, CAMKKbeta, BCL2, BCL-XL, AKT, ULK1, ULK2, ULK3, ULK4, DapK1, FIP200, TSC1, TSC2, STRAD, AMPK, Redd1, LKB, M025, PTEN, mTOR, Deptor, Rictor, Protor, PRAS40, LST8, Rheb, RAG A, RAG B, RAG C, RAG D, Raptor, PDK1, PI3K, IRS1, Insulin/IGF1 receptor, ERK, Rab40b, p53, DRAM1, NDFIP, MEK, RAF, SIN1, MAP4K3, Plac8, Dominant negative (DN) Rab7, Rab7, Dominant active (DA) Rab7, SLC7A5, or SLC3A2. In some embodiments, the autophagy modulator is ATG5. In certain embodiments, the autophagy modulator is an epigenetic regulator. Examples of epigenetic regulators include, but are not limited to histone methyltransferase (e.g., EZH2 (enhancer of zeste 2 polycomb repressive complex 2 subunit) or G9a), DNA methyltransferase (e.g., DNMT3), and histone deacetylases. In some embodiments, the ATG5 comprises an amino acid sequence at least 95% identical to the sequence of MTDDKDVLRDVWFGRIPTCFTLYQDEITEREAEPYYLLLPRVSYLTLVTDKVKKHF QKVMRQEDISEIWFEYEGTPLKWHYPIGLLFDLLASSSALPWNITVHFKSFPEKDLL HCPSKDAIEAHFMSCMKEADALKHKSQVINEMQKKDHKQLWMGLQNDRFDQFW AINRKLMEYPAEENGFRYIPFRIYQTTTERPFIQKLFRPVAADGQLHTLGDLLKEVC PSAIDPEDGEKKNQVMIHGIEPMLETPLQWLSEHLSYPDNFLHISIIPQPTD (SEQ ID NO: 1). In some embodiments, the ATG5 comprises an amino acid sequence at least 95% identical to the sequence of MTDDKDVLRDVWFGRIPTCFTLYQDEITEREAEPYYLLLPRVSYLTLVTDKVKKHF QKVMRQEDVSEIWFEYEGTPLKWHYPIGLLFDLLASSSALPWNITVHFKSFPEKDL LHCPSKDAVEAHFMSCMKEADALKHKSQVINEMQKKDHKQLWMGLQNDRFDQF WAINRKLMEYPPEENGFRYIPFRIYQTTTERPFIQKLFRPVAADGQLHTLGDLLREV CPSAVAPEDGEKRSQVMIHGIEPMLETPLQWLSEHLSYPDNFLHISIVPQPTD (SEQ ID NO: 2).

In some embodiments, the autophagy modulator is ATG7. In some embodiments, the ATG7 comprises an amino acid sequence at least 95% identical to the sequence of MAAATGDPGLSKLQFAPFSSALDVGFWHELTQKKLNEYRLDEAPKDIKGYYYNG DSAGLPARLTLEFSAFDMSAPTPARCCPAIGTLYNTNTLESFKTADKKLLLEQAAN EIWESIKSGTALENPVLLNKFLLLTFADLKKYHFYYWFCYPALCLPESLPLIQGPVG LDQRFSLKQIEALECAYDNLCQTEGVTALPYFLIKYDENMVLVSLLKHYSDFFQGQ RTKITIGVYDPCNLAQYPGWPLRNFLVLAAHRWSSSFQSVEVVCFRDRTMQGARD VAHSIIFEVKLPEMAFSPDCPKAVGWEKNQKGGMGPRMVNLSECMDPKRLAESSV DLNLKLMCWRLVPTLDLDKVVSVKCLLLGAGTLGCNVARTLMGWGVRHITFVD NAKISYSNPVRQPLYEFEDCLGGGKPKALAAADRLQKIFPGVNARGFNMSIPMPGH PVNFSSVTLEQARRDVEQLEQLIESHDVVFLLMDTRESRWLPAVIAASKRKLVINA ALGFDTFVVMRHGLKKPKQQGAGDLCPNHPVASADLLGSSLFANIPGYKLGCYFC NDVVAPGDSTRDRTLDQQCTVSRPGLAVIAGALAVELMVSVLQHPEGGYAIASSS DDRMNEPPTSLGLVPHQIRGFLSRFDNVLPVSLAFDKCTACSSKVLDQYEREGFNF LAKVFNSSHSFLEDLTGLTLLHQETQAAEIWDMSDDETI (SEQ ID NO: 3).

In some embodiments, the ATG7 comprises an amino acid sequence at least 95% identical to the sequence of MGDPGLAKLQFAPFNSALDVGFWHELTQKKLNEYRLDEAPKDIKGYYYNGDSAG LPTRLTLEFSAFDMSASTPAHCCPAMGTLHNTNTLEAFKTADKKLLLEQSANEIW EAIKSGAALENPMLLNKFLLLTFADLKKYHFYYWFCCPALCLPESIPLIRGPVSLDQ RLSPKQIQALEHAYDDLCRAEGVTALPYFLFKYDDDTVLVSLLKHYSDFFQGQRT KITVGVYDPCNLAQYPGWPLRNFLVLAAHRWSGSFQSVEVLCFRDRTMQGARDV THSIIFEVKLPEMAFSPDCPKAVGWEKNQKGGMGPRMVNLSGCMDPKRLAESSV DLNLKLMCWRLVPTLDLDKVVSVKCLLLGAGTLGCNVARTLMGWGVRHVTFV DNAKISYSNPVRQPLYEFEDCLGGGKPKALAAAERLQKIFPGVNARGFNMSIPMP GHPVNFSDVTMEQARRDVEQLEQLIDNHDVIFLLMDTRESRWLPTVIAASKRKLV INAALGFDTFVVMRHGLKKPKQQGAGDLCPSHLVAPADLGSSLFANIPGYKLGCY FCNDVVAPGDSTRDRTLDQQCTVSRPGLAVIAGALAVELMVSVLQHPEGGYAIAS SSDDRMNEPPTSLGLVPHQIRGFLSRFDNVLPVSLAFDKCTACSPKVLDQYEREGF TFLAKVFNSSHSFLEDLTGLTLLHQETQAAEIWDMSDEETV (SEQ ID NO: 4).

In various embodiments of the above methods, the vector is a lentiviral vector.

### BRIEF DESCRIPTION OF THE DRAWINGS

The foregoing will be apparent from the following more particular description of example embodiments, as illustrated in the accompanying drawings.

FIG. 1 shows the results of cell sorting analysis of (i) BMCA T-cells that ectopically express, via a lentiviral vector, ATG5 and the mCherry marker, and (ii) BMCA T-cells that ectopically express, via a lentiviral vector, ATG7 and GFP. Efficient expression of ATG5 and ATG7 was observed in these cells.

FIG. 2 shows the relative expression of ATG5 and ATG7 in BCMA CAR T-cells. BMCA T-cells that ectopically express ATG5 and ATG7 via a lentiviral vector were compared with those ectopically expressing a scrambled vector. As compared to cells expressing the scrambled vector (“Mock”), there was an approximately 8-fold increase in ATG5 gene expression and an approximately 14-fold increase of ATG7 gene expression.

FIG. 3 shows the results of a comparison of the degree of expansion of BCMA-CAR T cells expressing (i) BCMA-CAR alone, (ii) BCMA-CAR plus ATG5, and (iii) BCMA-CAR plus ATG7. The cells were stimulated with BCMA-expressing H929 cells at days 0, 7, 14, and 21. The repeated stimulation did increase the number of T cells expressing BCMA-CAR alone. However, there was a much larger comparable increase in cells expressing BCMA-CAR with either of ATG5 and ATG7. This data indicates that ectopic expression of ATG5 or ATG7 could enhance BCMA CAR-T expansion upon repeated tumor antigenic stimulation. Arrow indicates each stimulation.

FIG. 4 shows that tumor antigen drives T cell proliferation of BCMA CAR-T expressing ATG5 or ATG7. Untransduced T cells (Mock) were included as a control for non-specific cell expansion. BCMA-CAR cells were also used as a control. The left portion of the graph shows the absolute fold increase of the different cells observed upon stimulation by BCMA-expressing H929 cells. Greater expansion was seen in BCMA CAR cells expressing ATG5 or ATG7. The right portion of the graph shows the absolute fold increase of the different cells observed upon stimulation with IL-2, with no significant increased expansion in BCMA CAR cells expressing ATG5 or ATG7. The results show that overexpression of ATG5 or ATG7 augments tumor antigen-driven T proliferation, but not cytokine-induced proliferation.

FIG. 5 shows that ectopic expression of ATG5 and ATG7 does not impair CAR T cell cytotoxic activity. The cytotoxic capacity of BCMA-CAR cells (transduced with ATG5 or ATG7) was measured after overnight co-culture with luciferase expressing H929, along with untransduced mock transfected cells. Overexpression of ATG5 or ATG7 does not significantly impact CAR T cytotoxic capability.

FIG. 6 (top panel) shows flow cytometry plots of the expression of IFN-γ and TNF-α in each of BCMA-CAR cells (left), BCMA-CAR cells ectopically expressing ATG5 (middle), and BCMA-CAR cells ectopically expressing ATG7 (right). The BCMA-CAR⁺, BCMA-CAR⁺ATG5⁺, and BCMA-CAR⁺ATG7⁺ populations are determined as shown in FIG. 1. The top panel of FIG. 6 shows intracellular staining with IFN-γ and TNF-α. Quadrant gates split the analyzed cells into four adjacent, discrete sub-populations. The upper left quadrant represents the TNF⁺IFN⁻ population, the upper right quadrant represents the TNF⁺IFN⁺ population, the lower right quadrant represents the TNF⁻IFN⁺ population, and the lower left quadrant represents the TNF⁻IFN⁺ population. The bottom panel shows three bar graphs of the fraction of IFN-γ⁺, TNF-α⁺ and IFN-γ⁺ TNF-α⁺ cells. The white bar represents BCMA-CAR cells, the gray bar represents BCMA-CAR cells ectopically expressing ATG5, and the black bar represents BCMA-CAR cells ectopically expressing ATG7. The data indicate that overexpression of ATG5 and ATG7 could significantly increase CAR T cell production of effector cytokine IFN-γ and TNF-α.

FIG. 7 shows flow cytometry plots of the expression of CD45RO and CCR7 (upper panel dot plots), and CD27 (middle panel histograms) in each of BCMA-CAR cells (left), BCMA-CAR cells ectopically expressing ATG5 (middle), and BCMA-CAR cells ectopically expressing ATG7 (right). The bottom panel shows three bar graphs of the fraction of CD45RO⁻CCR7⁺ (naïve like), CD45RO⁺CCR7⁺ (central memory, Tcm) and CD27-expressing cells. The Pan-T cells were stimulated with CD3/CD28 Ab in the presence of IL-2 (50 U/ml), followed by lentiviral transduction of BCMA-CAR and ATG5 or ATG7 and then analyzed at day 10 after stimulation. The data indicate that overexpression of ATG5 and ATG7 does not significantly affect CAR-T differentiation during initial activation.

FIG. 8 shows four bar graphs of the fraction of CD45RO⁻CCR7⁺ (naïve like), CD45RO⁺CCR7⁺ (Tcm), CD45RO⁺CCR7⁻ (effector, Te) and CD27-expressing cells. The cells were stimulated with BCMA-expressing H929 cells at days 0, 7, 14, and 21. The repeated stimulation induced CAR-T cell differentiation into effector T cells and reduced their expression of CD27. However, CAR T cells expressing ATG5 had a naïve-like phenotype which differ from T cells expressing BCMA-CAR alone whose repertoires were dominated by effector/effector memory cells. In addition, the expression of CD27 was significantly increased in cells expressing BCMA-CAR with either of ATG5 and ATG7. This data indicated that ectopic expression of ATG5 or ATG7 could delay CAR-T effector differentiation but sustain CAR-T memory phenotype upon repetitive stimulation.

FIG. 9 shows flow cytometry plots (upper panel) of the expression of Granzyme B (GZMB) and Perforin (PRF) in each of BCMA-CAR cells (left), BCMA-CAR cells ectopically expressing ATG5 (middle), and BCMA-CAR cells ectopically expressing ATG7 (right). The bottom panel shows three bar graphs of the fraction of GZMB⁺, PRF⁺ and GZMB⁺PRF⁺ cells. The white bar represents BCMA-CAR cells, the black bar represents BCMA-CAR cells ectopically expressing ATG5, and the grey bar represents BCMA-CAR cells ectopically expressing ATG7. The data indicated that overexpression of ATG5 and ATG7 did not impair CAR T cell production of cytotoxic molecules GZMB or PRF.

FIG. 10 shows the representative 02 consumption rates (OCR) of Jurkat cells (transduced with or without ATG5) responding to a schematic of the mitochondrial stress test using the extracellular flux analyzer (upper panel). OCR was measured prior to the addition of drugs (basal OCR) and then following the addition of the indicated drugs. Reduction in OCR after oligomycin indicated the amount of 02 consumed for mitochondrial ATP generation. Administration of the oxidative phosphorylation uncoupler carbonyl cyanide-4-(trifluoromethoxy)phenylhydrazone (FCCP) allowed H+ back into the matrix independent of the ATP synthase; the cells attempted to maintain the chemiosmotic gradient after FCCP by moving H+ back out to the intermembrane space, which required the use of the electron transport chain (ETC) and the consumption of 02 as the final electron acceptor. After FCCP administration, the maximum capacity of the mitochondria to use oxidative phosphorylation (OXPHOS) was revealed. Spare respiratory capacity (SRC) is the difference between maximal OCR and basal OCR. Rotenone and Antimycin A administered together rendered a complete shutdown of the ETC, and thus mitochondrial oxygen consumption. The bottom panel shows four bar graphs of the level of basal OCR, maximal OCR and Spared respiration capacity and ATP. The white bar represents Jurkat cells and the black bar represents Jurkat cells ectopically expressing ATG5. The data indicate that overexpression of ATG5 significantly augmented cell mitochondrial function.

### DETAILED DESCRIPTION

A description of example embodiments follows.

The disclosure also provides related nucleic acids, recombinant expression vectors, host cells, populations of cells, antibodies, or antigen binding portions thereof, and pharmaceutical compositions relating to the immune cells and CAR-expressing immune cells of the invention.

Several aspects of the invention are described below, with reference to examples for illustrative purposes only. It should be understood that numerous specific details, relationships, and methods are set forth to provide a full understanding of the invention. One having ordinary skill in the relevant art, however, will readily recognize that the invention can be practiced without one or more of the specific details or practiced with other methods, protocols, reagents, cell lines and animals. The present invention is not limited by the illustrated ordering of acts or events, as some acts may occur in different orders and/or concurrently with other acts or events. Furthermore, not all illustrated acts, steps or events are required to implement a methodology in accordance with the present invention. Many of the techniques and procedures described, or referenced herein, are well understood and commonly employed using conventional methodology by those skilled in the art.

Unless otherwise defined, all terms of art, notations and other scientific terms or terminology used herein are intended to have the meanings commonly understood by those of skill in the art to which this invention pertains. In some cases, terms with commonly understood meanings are defined herein for clarity and/or for ready reference, and the inclusion of such definitions herein should not necessarily be construed to represent a substantial difference over what is generally understood in the art. It will be further understood that terms, such as those defined in commonly-used dictionaries, should be interpreted as having a meaning that is consistent with their meaning in the context of the relevant art and/or as otherwise defined herein.

### Definitions

Autophagy or “self-eating” is a biological process in which internal components of a cell are degraded in bulk in the lysosome. Autophagy is the only known means for large scale degradation and clearance of organelles and protein aggregates. Autophagy is used to present antigens, recycle amino acids from damaged proteins, degrade defunct organelles, and generate metabolites for energetic requirements. Macroautophagy (herein referred to as autophagy) was first described in Saccharomyces cerevisiae. 15 genes required for autophagy were identified (ATG1-15) in Saccharomyces cerevisiae, and have been found to be conserved in higher eukaryotes, including mammals. An “autophagy modulator” is a protein that increases or decreases autophagy. ATG5 is an example of an autophagy modulator that upregulates or increases the extent of autophagy in a cell. ATG7 is another example of an autophagy modulator that upregulates or increases the extent of autophagy in a cell. Without wishing to be bound by theory, ATG5 protein conjugates with ATG12 protein to facilitate the formation of autophagosome membranes. ATG7 regulates autophagosome assembly as well by activating ATG12 and ATG5. Ubiquitination is a means by which ATG5 and ATG7 signal other autophagy proteins to act in the autophagy pathway, or autophagy cascade.

The term “immune cell” refers to lymphocytes and other cells of the immune system. The term “immune cell” is used interchangeably with the term “immunoresponsive cell”. T cells and natural killer cells are two exemplary immune cells. Tumor infiltrating lymphocytes are another type of exemplary immune cell. Tumor-infiltrating lymphocytes are immune cells that have moved from the peripheral blood into a tumor. These lymphocytes may have the ability to attack a tumor. The function of tumor-infiltrating lymphocytes may be altered in a tumor environment. In the context of cancer therapy, tumor-infiltrating lymphocytes are removed from the tumor of a patient, and then treated (e.g., contacted with substances and/or engineered in the laboratory). Treatment may be effective to activate the lymphocytes for improved efficacy to target and destroy cancer cells in the patient.

The terms “T cell” and “T lymphocyte” are interchangeable and used synonymously herein. As used herein, T cell includes thymocytes, naive T lymphocytes, immature T lymphocytes, mature T lymphocytes, resting T lymphocytes, or activated T lymphocytes. A T cell can be a T helper (Th) cell, for example a T helper 1 (Th1) or a T helper 2 (Th2) cell. The T cell can be a helper T cell (HTL; CD4+ T cell) CD4+ T cell, a cytotoxic T cell (CTL; CD8+ T cell), a tumor infiltrating cytotoxic T cell (TIL; CD8+ T cell), CD4+CD8+ T cell, or any other subset of T cells. Other illustrative populations of T cells suitable for use in particular embodiments include naive T cells and memory T cells. Also included are “NKT cells”, which refer to a specialized population of T cells that express a semi-invariant αβ T-cell receptor, but also express a variety of molecular markers that are typically associated with NK cells, such as NK1.1. NKT cells include NK1.1+ and NK1.1-, as well as CD4+, CD4-, CD8+ and CD8− cells. The TCR on NKT cells is unique in that it recognizes glycolipid antigens presented by the MHC I-like molecule CD Id. NKT cells can have either protective or deleterious effects due to their abilities to produce cytokines that promote either inflammation or immune tolerance. Also included are “gamma-delta T cells (γδ T cells),” which refer to a specialized population that to a small subset of T cells possessing a distinct TCR on their surface, and unlike the majority of T cells in which the TCR is composed of two glycoprotein chains designated α- and β-TCR chains, the TCR in γδ T cells is made up of a γ-chain and a δ-chain. γδ T cells can play a role in immunosurveillance and immunoregulation, and were found to be an important source of IL-17 and to induce robust CD8+ cytotoxic T cell response. Also included are “regulatory T cells” or “Tregs”, which refer to T cells that suppress an abnormal or excessive immune response and play a role in immune tolerance. Tregs cells are typically transcription factor Foxp3-positive CD4+ T cells and can also include transcription factor Foxp3-negative regulatory T cells that are IL-10-producing CD4+ T cells.

The terms “natural killer cell” and “NK cell” are used interchangeably and synonymously herein. As used herein, NK cell refers to a differentiated lymphocyte with a CD 16+CD56+ and/or CD57+ TCR− phenotype. NKs are characterized by their ability to bind to and kill cells that fail to express “self” MHC/HLA antigens by the activation of specific cytolytic enzymes, the ability to kill tumor cells or other diseased cells that express a ligand for NK activating receptors, and the ability to release protein molecules called cytokines that stimulate or inhibit the immune response.

The term “chimeric antigen receptor” or “CAR” as used herein is defined as a cell-surface receptor comprising an extracellular target-binding domain, a transmembrane domain and an intracellular signaling domain, all in a combination that is not naturally found together on a single protein. This particularly includes receptors wherein the extracellular domain and the intracellular signaling domain are not naturally found together on a single receptor protein. Chimeric antigen receptors are intended primarily for use with lymphocyte such as T cells and natural killer (NK) cells.

As used herein, the term “antigen” refers to any agent (e.g., protein, peptide, polysaccharide, glycoprotein, glycolipid, nucleic acid, portions thereof, or combinations thereof) molecule capable of being bound by a T-cell receptor. An antigen is also able to provoke an immune response.

The term “host cell” means any cell that contains a heterologous nucleic acid. The heterologous nucleic acid can be a vector (e.g., an expression vector). For example, a host cell can be a cell from any organism that is selected, modified, transformed, grown, used or manipulated in any way, for the production of a substance by the cell, for example the expression by the cell of a gene, a DNA or RNA sequence, a protein or an enzyme. An appropriate host may be determined. For example, the host cell may be selected based on the vector backbone and the desired result. By way of example, a plasmid or cosmid can be introduced into a prokaryote host cell for replication of several types of vectors. Bacterial cells such as, but not limited to DH5α, JM109, and KCB, SURE® Competent Cells, and SOLOPACK Gold Cells, can be used as host cells for vector replication and/or expression. Additionally, bacterial cells such as E. coli LE392 could be used as host cells for phage viruses. Eukaryotic cells that can be used as host cells include, but are not limited to yeast (e.g., YPH499, YPH500 and YPH501), insects and mammals. Examples of mammalian eukaryotic host cells for replication and/or expression of a vector include, but are not limited to, HeLa, NIH3T3, Jurkat, 293, COS, CHO, Saos, and PC12.

Host cells of the present disclosure include T cells and natural killer cells that contain DNA or RNA sequences encoding autophagy modulators, the CAR and that express the CAR on the cell surface. Host cells may be used for enhancing T cell activity, natural killer cell activity, treatment of cancer, and treatment of autoimmune disease.

“Activation” or “stimulation” means to induce a change in the cells' biologic state by which the cells (e.g., T cells and NK cells) express activation markers, produce cytokines, undergo more autophagy, proliferate and/or become cytotoxic to target cells. All these changes can be produced by primary stimulatory signals. Co-stimulatory signals can amplify the magnitude of the primary signals and suppress cell death following initial stimulation resulting in a more durable activation state and thus a higher cytotoxic capacity. A “co-stimulatory signal” refers to a signal, which in combination with a primary signal, such as TCR/CD3 ligation, leads to T cell and/or NK cell proliferation and/or upregulation or downregulation of key molecules. Activation or stimulation of autophagy can occur by ectopic expression of one or more autophagy modulators that increase the rate or extent of autophagy, e.g., ATG5 or ATG7.

The term “proliferation” refers to an increase in cell division, either symmetric or asymmetric division of cells. The term “expansion” refers to the outcome of cell division and cell death.

The term “differentiation” refers to a method of decreasing the potency or proliferation of a cell or moving the cell to a more developmentally restricted state.

The terms “express” and “expression” mean allowing or causing the information in a gene or DNA sequence to become produced, for example producing a protein by activating the cellular functions involved in transcription and translation of a corresponding gene or DNA sequence. A DNA sequence is expressed in or by a cell to form an “expression product” such as a protein. The expression product itself, e.g., the resulting protein, may also be said to be “expressed” by the cell. An expression product can be characterized as intracellular, extracellular or transmembrane.

The term “transfection” means the introduction of a “foreign” (i.e., extrinsic or extracellular) nucleic acid into a cell using recombinant DNA technology. The term “genetic modification” means the introduction of a “foreign” (i.e., extrinsic or extracellular) gene, DNA or RNA sequence to a host cell, so that the host cell will express the introduced gene or sequence to produce a desired substance, typically a protein or enzyme coded by the introduced gene or sequence. The introduced gene or sequence may also be called a “cloned” or “foreign” gene or sequence, may include regulatory or control sequences operably linked to polynucleotide encoding the chimeric antigen receptor, such as start, stop, promoter, signal, secretion, or other sequences used by a cell's genetic machinery. The gene or sequence may include nonfunctional sequences or sequences with no known function. A host cell that receives and expresses introduced DNA or RNA has been “genetically engineered.” The DNA or RNA introduced to a host cell can come from any source, including cells of the same genus or species as the host cell, or from a different genus or species.

The term “transduction” means the introduction of a foreign nucleic acid into a cell using a viral vector.

The term “regulatory element” refers to any cis-acting genetic element that controls some aspect of the expression of nucleic acid sequences. In some embodiments, the term “promoter” comprises essentially the minimal sequences required to initiate transcription. In some embodiments, the term “promoter” includes the sequences to start transcription, and in addition, also include sequences that can upregulate or downregulate transcription, commonly termed “enhancer elements” and “repressor elements”, respectively.

As used herein, the term “operatively linked,” and similar phrases, when used in reference to nucleic acids or amino acids, refer to the operational linkage of nucleic acid sequences or amino acid sequence, respectively, placed in functional relationships with each other. For example, an operatively linked promoter, enhancer elements, open reading frame, 5′ and 3′ UTR, and terminator sequences result in the accurate production of a nucleic acid molecule (e.g., RNA). In some embodiments, operatively linked nucleic acid elements result in the transcription of an open reading frame and ultimately the production of a polypeptide (i.e., expression of the open reading frame). As another example, an operatively linked peptide is one in which the functional domains are placed with appropriate distance from each other to impart the intended function of each domain.

By “enhance” or “promote,” or “increase” or “expand” or “improve” refers generally to the ability of a composition contemplated herein to produce, elicit, or cause a greater physiological response (i.e., downstream effects) compared to the response caused by either vehicle or a control molecule/composition. A measurable physiological response may include an increase in T cell expansion, activation, effector function, persistence, and/or an increase in cancer cell death killing ability, among others apparent from the understanding in the art and the description herein. In certain embodiments, an “increased” or “enhanced” amount can be a “statistically significant” amount, and may include an increase that is 1.1, 1.2, 1.5, 2, 3, 4, 5, 6, 7, 8, 9, 10, 15, 20, 30 or more times (e.g., 500, 1000 times) (including all integers and decimal points in between and above 1, e.g., 1.5, 1.6, 1.7. 1.8, etc.) the response produced by vehicle or a control composition.

By “decrease” or “lower,” or “lessen,” or “reduce,” or “abate” refers generally to the ability of composition contemplated herein to produce, elicit, or cause a lesser physiological response (i.e., downstream effects) compared to the response caused by either vehicle or a control molecule/composition. In certain embodiments, a “decrease” or “reduced” amount can be a “statistically significant” amount, and may include a decrease that is 1.1, 1.2, 1.5, 2, 3, 4, 5, 6, 7, 8, 9, 10, 15, 20, 30 or more times (e.g., 500, 1000 times) (including all integers and decimal points in between and above 1, e.g., 1.5, 1.6, 1.7. 1.8, etc.) the response (reference response) produced by vehicle, a control composition, or the response in a particular cell lineage.

The term “effective” applied to dose or amount refers to that quantity of a compound or pharmaceutical composition that is sufficient to result in a desired activity upon administration to a subject in need thereof. Note that when a combination of active ingredients is administered, the effective amount of the combination may or may not include amounts of each ingredient that would have been effective if administered individually. The exact amount required will vary from subject to subject, depending on the species, age, and general condition of the subject, the severity of the condition being treated, the particular drug or drugs employed, the mode of administration, and the like.

The phrase “pharmaceutically acceptable”, as used in connection with compositions described herein, refers to molecular entities and other ingredients of such compositions that are physiologically tolerable and do not typically produce untoward reactions when administered to a mammal (e.g., a human). Preferably, the term “pharmaceutically acceptable” means approved by a regulatory agency of the Federal or a state government or listed in the U.S. Pharmacopeia or other generally recognized pharmacopeia for use in mammals, and more particularly in humans.

The term “protein” is used herein encompasses all kinds of naturally occurring and synthetic proteins, including protein fragments of all lengths, fusion proteins and modified proteins, including without limitation, glycoproteins, as well as all other types of modified proteins (e.g., proteins resulting from phosphorylation, acetylation, myristoylation, palmitoylation, glycosylation, oxidation, formylation, amidation, polyglutamylation, ADP-ribosylation, pegylation, biotinylation, etc.).

The terms “nucleic acid”, “nucleotide”, and “polynucleotide” encompass both DNA and RNA unless specified otherwise. By a “nucleic acid sequence” or “nucleotide sequence” is meant the nucleic acid sequence encoding an amino acid; these terms may also refer to the nucleic acid sequence including the portion coding for any amino acids added as an artifact of cloning, including any amino acids coded for by linkers.

The term “carrier” refers to a diluent, adjuvant, excipient, or vehicle with which the compound is administered. Such pharmaceutical carriers can be sterile liquids, such as water and oils, including those of petroleum, animal, vegetable or synthetic origin, such as peanut oil, soybean oil, mineral oil, sesame oil and the like. Water or aqueous solution saline solutions and aqueous dextrose and glycerol solutions are preferably employed as carriers, particularly for injectable solutions. Alternatively, the carrier can be a solid dosage form carrier, including but not limited to one or more of a binder (for compressed pills), a glidant, an encapsulating agent, a flavorant, and a colorant. Suitable pharmaceutical carriers are described in “Remington's Pharmaceutical Sciences” by E. W. Martin.

The term “about” or “approximately” includes being within a statistically meaningful range of a value. Such a range can be within an order of magnitude, preferably within 50%, more preferably within 20%, still more preferably within 10%, and even more preferably within 5% of a given value or range. The allowable variation encompassed by the term “about” or “approximately” depends on the particular system under study, and can be readily appreciated by one of ordinary skill in the art.

The terminology used herein is for the purpose of describing particular embodiments only and is not intended to be limiting. As used herein, the indefinite articles “a”, “an” and “the” should be understood to include plural reference unless the context clearly indicates otherwise.

Chimeric Antigen Receptors in Immune Cells with Modulated Autophagy

The present invention relates generally to the use of immune cells genetically modified to stably express a desired chimeric antigen receptor, and in which autophagy is modulated. In some immune cells, autophagy is activated or upregulated, e.g., by increasing the rate or extent of autophagy. Autophagy can be upregulated by ectopic expression of an autophagy modulator, such as ATG5 or ATG7. Autophagy modulators include, but are not limited to, ATG1, ATG2, ATG3, ATG4, ATG5, ATG6, ATG7, ATG8, ATG8, ATG10, ATG11, ATG12, ATG13, ATG14, ATG15, ATG16, ATG17, ATG18, ATG19, ATG20, ATG21, ATG22, ATG23, ATG24, ATG25, ATG26, ATG27, ATG28, ATG29, ATG30, ATG31, ATG101, LC3, RAB7, VPS15, VPS34, VPS35, LC3I, LC3II, UVRAG, Beclin1, Protor, CAMKKbeta, BCL2, BCL-XL, AKT, ULK1, ULK2, ULK3, ULK4, DapK1, FIP200, TSC1, TSC2, STRAD, AMPK, Redd1, LKB, M025, PTEN, mTOR, Deptor, Rictor, Protor, PRAS40, LST8, Rheb, RAG A, RAG B, RAG C, RAG D, Raptor, PDK1, PI3K, IRS1, Insulin/IGF1 receptor, ERK, Rab40b, p53, DRAM1, NDFIP, MEK, RAF, SIN1, MAP4K3, Plac8, Dominant negative (DN) Rab7, Rab7, Dominant active (DA) Rab7, SLC7A5, and SLC3A2. In certain embodiments, the autophagy modulator is an epigenetic regulator. Examples of epigenetic regulators include, but are not limited to histone methyltransferase (e.g., EZH2 (enhancer of zeste 2 polycomb repressive complex 2 subunit) or G9a), DNA methyltransferase (e.g., DNMT3), and histone deacetylases.

A chimeric antigen receptor (CAR) is an artificially constructed hybrid protein or polypeptide containing the antigen binding domains of an antibody (scFv) linked to T-cell signaling domains. Characteristics of CARs can include their ability to redirect T-cell specificity and reactivity toward a selected target in a non-MHC-restricted manner, exploiting the antigen-binding properties of monoclonal antibodies. The non-MHC-restricted antigen recognition gives T cells expressing CARs the ability to recognize antigens independent of antigen processing, thus bypassing a major mechanism of tumor evasion. Moreover, when expressed in T-cells, CARs advantageously do not dimerize with endogenous T cell receptor (TCR) alpha and beta chains. T cells expressing a CAR are referred to herein as CAR T cells, CAR-T cells or CAR modified T cells, and these terms are used interchangeably herein. The cell can be genetically modified to stably express an antibody binding domain on its surface, conferring novel antigen specificity that is MHC independent.

In some instances, the T cell is genetically modified to stably express a CAR that combines an antigen recognition domain of a specific antibody with an intracellular domain of the CD3-zeta chain or FcγRI protein into a single chimeric protein. In one embodiment, the stimulatory molecule is the zeta chain associated with the T cell receptor complex.

An “intracellular signaling domain,” as the term is used herein, refers to an intracellular portion of a molecule. It is the functional portion of the protein which acts by transmitting information within the cell to regulate cellular activity via defined signaling pathways by generating second messengers or functioning as effectors by responding to such messengers. The intracellular signaling domain generates a signal that promotes an immune effector function of the CAR containing cell, e.g., a CAR-T cell. Examples of immune effector function, e.g., in a CAR-T cell, include cytolytic activity and helper activity, including the secretion of cytokines.

In an embodiment, the intracellular signaling domain can comprise a primary intracellular signaling domain. Example primary intracellular signaling domains include those derived from the molecules responsible for primary stimulation, or antigen dependent simulation. In an embodiment, the intracellular signaling domain can comprise a co-stimulatory intracellular domain. Example co-stimulatory intracellular signaling domains include those derived from molecules responsible for co-stimulatory signals, or antigen independent stimulation. For example, in the case of a CAR-T, a primary intracellular signaling domain can comprise a cytoplasmic sequence of a T cell receptor, and a co-stimulatory intracellular signaling domain can comprise cytoplasmic sequence from co-receptor or co-stimulatory molecule.

A primary intracellular signaling domain can comprise a signaling motif which is known as an immunoreceptor tyrosine-based activation motif or ITAM. Examples of ITAM containing primary cytoplasmic signaling sequences include, but are not limited to, those derived from CD3-zeta, FcR gamma, FcR beta, CD3 gamma, CD3 delta, CD3 epsilon, CD5, CD22, CD79a, CD79b, and CD66d DAP10 and DAP12.

The term “zeta” or alternatively “zeta chain”, “CD3-zeta” or “TCR-zeta” is defined as the protein provided as GenBank Acc. No. BAG36664.1, or the equivalent residues from a nonhuman species, e.g., murine, rabbit, primate, mouse, rodent, monkey, ape and the like, and a “zeta stimulatory domain” or alternatively a “CD3-zeta stimulatory domain” or a “TCR-zeta stimulatory domain” is defined as the amino acid residues from the cytoplasmic domain of the zeta chain that are sufficient to functionally transmit an initial signal necessary for T cell activation. In one aspect, the cytoplasmic domain of zeta comprises residues 52 through 164 of GenBank Acc. No. BAG36664.1 or the equivalent residues from a non-human species, e.g., mouse, rodent, monkey, ape and the like, that are functional orthologs thereof.

The term “co-stimulatory molecule” refers to the cognate binding partner on a T cell that specifically binds with a co-stimulatory ligand, thereby mediating a co-stimulatory response by the T cell, such as, but not limited to, proliferation. Co-stimulatory molecules are cell surface molecules other than antigen receptors or their ligands that are required for an efficient immune response. Co-stimulatory molecules include, but are not limited to an MHC class 1 molecule, BTLA and a Toll ligand receptor, as well as OX40, CD2, CD27, CD28, CD S, ICAM-1, LFA-1 (CD11a/CD18) and 4-1BB (CD137).

A co-stimulatory intracellular signaling domain can be the intracellular portion of a co-stimulatory molecule. A co-stimulatory molecule can be represented in the following protein families: TNF receptor proteins, Immunoglobulin-like proteins, cytokine receptors, integrins, signaling lymphocytic activation molecules (SLAM proteins), and activating NK cell receptors. Examples of such molecules include CD27, CD28, 4-1BB (CD137), OX40, GITR, CD30, MyD88, CD40, ICOS, BAFFR, HVEM, lymphocyte function-associated antigen-1 (LFA-1), CD2, CD7, LIGHT, NKG2C, SLAMF7, NKp80, CD160, B7-H3, and a ligand that specifically binds with CD83, and the like.

The intracellular signaling domain can comprise the entire intracellular portion, or the entire native intracellular signaling domain, of the molecule from which it is derived, or a functional fragment thereof.

The term “4-1BB” or alternatively “CD137” refers to a member of the TNFR superfamily with an amino acid sequence provided as GenBank Acc. No. AAA62478.2, or the equivalent residues from a nonhuman species, e.g., mouse, rodent, monkey, ape and the like; and a “4-1BB co-stimulatory domain” is defined as amino acid residues 214-255 of GenBank accession no. AAA62478.2, or the equivalent residues from a non-human species, e.g., mouse, rodent, monkey, ape and the like.

In one embodiment, a transmembrane domain that naturally is associated with one of the domains in the CAR is used. In another embodiment, the transmembrane domain can be selected or modified by amino acid substitution to avoid binding of such domains to the transmembrane domains of the same or different surface membrane proteins to minimize interactions with other members of the receptor complex. In one example embodiment, the transmembrane domain comprises the CD8α hinge domain.

In some embodiments, the cytoplasmic signaling domain further comprises one or more functional signaling domains derived from at least one co-stimulatory molecule as defined herein. In one embodiment, the co-stimulatory molecule is chosen from 4-1BB (i.e., CD137), CD27, CD3-zeta and/or CD28. In some embodiments, the CAR comprises an intracellular hinge domain comprising CD8 and an intracellular T cell receptor signaling domain comprising CD28, 4-1BB, and CD3-zeta. In another embodiment, the CAR comprises an intracellular hinge domain and an intracellular T cell receptor signaling domain comprising CD28, 4-1BB, and CD3-zeta, wherein the hinge domain comprises all or part of the extracellular region of CD8, CD4 or CD28; all or part of an antibody constant region; all or part of the FcγRIIIa receptor, an IgG hinge, an IgM hinge, an IgA hinge, an IgD hinge, an IgE hinge, or an Ig hinge. The IgG hinge may be from IgG1, IgG2, IgG3, IgG4, IgM1, IgM2, IgA1, IgA2, IgD, IgE, or a chimera thereof.

CARs described herein provide recombinant polypeptide constructs comprising at least an extracellular antigen binding domain, a transmembrane domain and an intracellular signaling domain (also referred to herein as “a cytoplasmic signaling domain”) comprising, e.g., a functional signaling domain derived from a stimulatory molecule as defined below

In one embodiment, the CAR comprises a chimeric fusion protein comprising an extracellular antigen recognition domain, a transmembrane domain and an intracellular signaling domain comprising a functional signaling domain derived from a stimulatory molecule. In one embodiment, the CAR comprises a chimeric fusion protein comprising an extracellular antigen recognition domain, a transmembrane domain and an intracellular signaling domain comprising a functional signaling domain derived from a co-stimulatory molecule and a functional signaling domain derived from a stimulatory molecule. In one embodiment, the CAR comprises a chimeric fusion protein comprising an extracellular antigen recognition domain, a transmembrane domain and an intracellular signaling domain comprising at least two functional signaling domains derived from one or more co-stimulatory molecule(s) and a functional signaling domain derived from a stimulatory molecule.

The disclosure further provides variants, e.g., functional variants, of the CARs, nucleic acids, polypeptides, and proteins described herein. “Variant” refers to a polypeptide or a polynucleotide that differs from a reference polypeptide or a reference polynucleotide by one or more modifications for example, substitutions, insertions or deletions. The term “functional variant” as used herein refers to a CAR, polypeptide, or protein having substantial or significant sequence identity or similarity to a parent CAR, polypeptide, or protein, which functional variant retains the biological activity of the CAR, polypeptide, or protein for which it is a variant. Functional variants encompass, e.g., those variants of the CAR, polypeptide, or protein described herein (the parent CAR, polypeptide, or protein) that retain the ability to recognize target cells to a similar extent, the same extent, or to a higher extent, as the parent CAR, polypeptide, or protein. In reference to the parent CAR, polypeptide, or protein, the functional variant can, for example, be at least about 30%, about 40%, about 50%, about 60%, about 75%, about 80%, about 85%, about 90%, about 91%, about 92%, about 93%, about 94%, about 95%, about 96%, about 97%, about 98%, about 99% or more identical in amino acid sequence to the parent CAR, polypeptide, or protein.

The CARs, polypeptides, and proteins of embodiments of the disclosure (including functional portions and functional variants) can be of any length, i.e., can comprise any number of amino acids, provided that the CARs, polypeptides, or proteins (or functional portions or functional variants thereof) retain their biological activity, e.g., the ability to specifically bind to an antigen, detect diseased cells (e.g., cancer cells) in a host, or treat or prevent disease in a host, etc. For example, the polypeptide can be about 50 to about 5000 amino acids long, such as about 50, about 70, about 75, about 100, about 125, about 150, about 175, about 200, about 225, about 250, about 275, about 300, about 325, about 350, about 375, about 400, about 425, about 450, about 475, about 500, about 525, about 550, about 575, about 600, about 625, about 650, about 675, about 700, about 725, about 750, about 775, about 800, about 825, about 850, about 875, about 900, about 925, about 950, about 975, about 1000 or more amino acids in length. The polypeptides of the invention also include oligopeptides.

The autophagy modulators, CARs, polypeptides, and proteins of embodiments described herein (including functional portions and functional variants of the invention) can comprise synthetic amino acids in place of one or more naturally-occurring amino acids. Such synthetic amino acids are known in the art, and include, for example, aminocyclohexane carboxylic acid, norleucine, α-amino n-decanoic acid, homoserine, S-acetylaminomethyl-cysteine, trans-3- and trans-4-hydroxyproline, 4-aminophenylalanine, 4-nitrophenylalanine, α-(2-amino-2-norbornane)-carboxylic acid, α,γ-diaminobutyric acid, α,β-diaminopropionic acid, homophenylalanine, 4-chlorophenylalanine, 4-carboxyphenylalanine, β-phenyl serine β-hydroxyphenylalanine, phenylglycine, α-naphthylalanine, cyclohexylalanine, cyclohexylglycine, N′-benzyl-N′-methyl-lysine, N′,N′-dibenzyl-lysine, 6-hydroxylysine, ornithine, α-aminocyclopentane carboxylic acid, α-aminocyclohexane carboxylic acid, α-aminocycloheptane carboxylic acid, indoline-2-carboxylic acid, 1,2,3,4-tetrahydroisoquinoline-3-carboxylic acid, aminomalonic acid, aminomalonic acid monoamide, and α-tert-butylglycine.

The autophagy modulators, CARs, polypeptides, and proteins of embodiments described herein (including functional portions and functional variants) can be subject to post-translational modifications. They can be glycosylated, esterified, N-acylated, amidated, carboxylated, phosphorylated, esterified, cyclized via, e.g., a disulfide bridge, or converted into an acid addition salt. In some embodiments, they are dimerized or polymerized, or conjugated.

The autophagy modulators, CARs, polypeptides, and/or proteins of embodiments of the invention (including functional portions and functional variants thereof) can be obtained by methods known in the art. Suitable methods of de novo synthesizing polypeptides and proteins are described in references, such as Chan et al., Fmoc Solid Phase Peptide Synthesis, Oxford University Press, Oxford, United Kingdom, 2000; Peptide and Protein Drug Analysis, ed. Reid, R., Marcel Dekker, Inc., 2000; and Epitope Mapping, ed. Westwood et al., Oxford University Press, Oxford, United Kingdom, 2001. Also, polypeptides and proteins can be recombinantly produced using the nucleic acids described herein using standard recombinant methods. See, for instance, Sambrook et al., Molecular Cloning: A Laboratory Manual, 3rd ed., Cold Spring Harbor Press, Cold Spring Harbor, N.Y. 2001; and Ausubel et al., Current Protocols in Molecular Biology, Greene Publishing Associates and John Wiley & Sons, N Y, 1994. Further, some of the autophagy modulators, CARs, polypeptides, and proteins of the invention (including functional portions and functional variants thereof) can be isolated and/or purified from a source, such as a plant, a bacterium, an insect, a mammal, etc. Methods of isolation and purification are known in the art. Alternatively, the autophagy modulators, CARs, polypeptides, and/or proteins described herein (including functional portions and functional variants thereof) can be commercially synthesized. In this respect, the autophagy modulators, CARs, polypeptides, and proteins can be synthetic, recombinant, isolated, and/or purified.

Examples of modified nucleotides that can be used to generate the recombinant nucleic acids utilized to produce the polypeptides described herein include, but are not limited to, 5-fluorouracil, 5-bromouracil, 5-chlorouracil, 5-iodouracil, hypoxanthine, xanthine, 4-acetylcytosine, 5-(carboxyhydroxymethyl) uracil, carboxymethylaminomethyl-2-thiouridine, 5-carboxymethylaminomethyluracil, dihydrouracil, N⁶-substituted adenine, 7-methylguanine, 5-methylaminomethyluracil, 5-methoxyaminomethyl-2-thiouracil, beta-D-mannosylqueosine, 5″-methoxycarboxymethyluracil, 5-methoxyuracil, 2-methylthio-N⁶-isopentenyladenine, uracil-5-oxyacetic acid (v), wybutoxosine, pseudouracil, queuosine, beta-D-galactosylqueosine, inosine, N⁶-isopentenyladenine, 1-methylguanine, 1-methylinosine, 2,2-dimethylguanine, 2-methyladenine, 2-methylguanine, 3-methylcytosine, 5-methylcytosine, 2-thiocytosine, 5-methyl-2-thiouracil, 2-thiouracil, 4-thiouracil, 5-methyluracil, uracil-5-oxyacetic acid methylester, 3-(3-amino-3-N-2-carboxypropyl) uracil, and 2,6-diaminopurine.

The nucleic acid can comprise any isolated or purified nucleotide sequence which encodes any of the autophagy modulators, CARs, polypeptides, or proteins, or functional portions or functional variants thereof. Alternatively, the nucleotide sequence can comprise a nucleotide sequence which is degenerate to any of the sequences or a combination of degenerate sequences.

Some embodiments of the invention also provide an isolated or purified nucleic acid comprising a nucleotide sequence which is complementary to the nucleotide sequence of any of the nucleic acids described herein or a nucleotide sequence which hybridizes under stringent conditions to the nucleotide sequence of any of the nucleic acids described herein.

The nucleotide sequence which hybridizes under stringent conditions may hybridize under high stringency conditions. By “high stringency conditions” is meant that the nucleotide sequence specifically hybridizes to a target sequence (the nucleotide sequence of any of the nucleic acids described herein) in an amount that is detectably stronger than non-specific hybridization. High stringency conditions include conditions which would distinguish a polynucleotide with an exact complementary sequence, or one containing only a few scattered mismatches from a random sequence that happened to have a few small regions (e.g., 3-12 bases) that matched the nucleotide sequence. Such small regions of complementarity are more easily melted than a full-length complement of 14-17 or more bases, and high stringency hybridization makes them easily distinguishable. Relatively high stringency conditions would include, for example, low salt and/or high temperature conditions, such as provided by about 0.02-0.1 M NaCl or the equivalent, at temperatures of about 50-70° C. Such high stringency conditions tolerate little, if any, mismatch between the nucleotide sequence and the template or target strand, and are particularly suitable for detecting expression of any of the CARs described herein. It is generally appreciated that conditions can be rendered more stringent by the addition of increasing amounts of formamide.

In an embodiment, the nucleic acids of the invention can be incorporated into a recombinant expression vector. The present disclosure provides recombinant expression vectors comprising any of the nucleic acids of the invention. As used herein, the term “recombinant expression vector” means a genetically-modified oligonucleotide or polynucleotide construct that permits the expression of an mRNA, protein, polypeptide, or peptide by a host cell, when the construct comprises a nucleotide sequence encoding the mRNA, protein, polypeptide, or peptide, and the vector is contacted with the cell under conditions sufficient to have the mRNA, protein, polypeptide, or peptide expressed within the cell. The vectors described herein are not naturally-occurring as a whole; however, parts of the vectors can be naturally-occurring. The described recombinant expression vectors can comprise any type of nucleotides, including, but not limited to DNA and RNA, which can be single-stranded or double-stranded, synthesized or obtained in part from natural sources, and which can contain natural, non-natural or altered nucleotides. The recombinant expression vectors can comprise naturally-occurring or non-naturally-occurring internucleotide linkages, or both types of linkages. The non-naturally occurring or altered nucleotides or internucleotide linkages do not hinder the transcription or replication of the vector.

The vector may comprise a nucleotide sequence encoding a chimeric antigen receptor (CAR), as well as a second nucleotide sequence encoding an autophagy modulator. Alternatively, one vector may comprise nucleotide sequence encoding a chimeric antigen receptor (CAR), with another vector comprising a nucleotide sequence encoding an autophagy modulator.

In an embodiment, the recombinant expression vector of the invention can be any suitable recombinant expression vector, and can be used to transform or transfect any suitable host. Suitable vectors include those designed for propagation and expansion or for expression or both, such as plasmids and viruses. The vector can be selected from the group consisting of the pUC series (Fermentas Life Sciences, Glen Burnie, Md.), the pBluescript series (Stratagene, La Jolla, Calif.), the pET series (Novagen, Madison, Wis.), the pGEX series (Pharmacia Biotech, Uppsala, Sweden), and the pEX series (Clontech, Palo Alto, Calif.). Bacteriophage vectors, such as λGT10, λGT11, λEMBL4, and λNM1149, λZapII (Stratagene) can be used. Examples of plant expression vectors include pBI01, pBI01.2, pBI121, pBI101.3, and pBIN19 (Clontech). Examples of animal expression vectors include pEUK-Cl, pMAM, and pMAMneo (Clontech). The recombinant expression vector may be a viral vector, e.g., a retroviral vector, e.g., a gamma retroviral vector.

In an embodiment, the recombinant expression vectors of the invention are prepared using standard recombinant DNA techniques described in, for example, Sambrook et al., supra, and Ausubel et al., supra. Constructs of expression vectors, which are circular or linear, can be prepared to contain a replication system functional in a prokaryotic or eukaryotic host cell. Replication systems can be derived, e.g., from ColEl, SV40, 2 μl plasmid, λ bovine papilloma virus, and the like.

The recombinant expression vector may comprise regulatory sequences, such as transcription and translation initiation and termination codons, which are specific to the type of host (e.g., bacterium, plant, fungus, or animal) into which the vector is to be introduced, as appropriate, and taking into consideration whether the vector is DNA- or RNA-based.

The recombinant expression vector can include one or more marker genes, which allow for selection of transformed or transfected hosts. Marker genes include biocide resistance, e.g., resistance to antibiotics, heavy metals, etc., complementation in an auxotrophic host to provide prototrophy, and the like. Suitable marker genes for the described expression vectors include, for instance, neomycin/G418 resistance genes, histidinol x resistance genes, histidinol resistance genes, tetracycline resistance genes, and ampicillin resistance genes.

The recombinant expression vector can comprise a native or normative promoter operably linked to the nucleotide sequence encoding the autophagy modulator, CAR, polypeptide, or protein (including functional portions and functional variants thereof), or to the nucleotide sequence which is complementary to or which hybridizes to the nucleotide sequence encoding the CAR, polypeptide, or protein. The selection of promoters, e.g., strong, weak, tissue-specific, inducible and developmental-specific, is within the ordinary skill of the artisan. Similarly, the combining of a nucleotide sequence with a promoter is also within the skill of the artisan. The promoter can be a non-viral promoter or a viral promoter, e.g., a cytomegalovirus (CMV) promoter, an RSV promoter, an SV40 promoter, or a promoter found in the long-terminal repeat of the murine stem cell virus.

The recombinant expression vectors can be designed for either transient expression, for stable expression, or for both. Also, the recombinant expression vectors can be made for constitutive expression or for inducible expression.

In certain embodiments, the promoter has an activity that is modulated by a small molecule. The promoter may become more active in the presence of a small molecule drug so that the expression of an autophagy modulator (e.g., ATG5 or ATG7) operatively linked to the promoter increases when the small molecule drug is administered to the subject or to the immune cells. Conversely, the promoter may become less active in the presence of a small molecule drug so that the expression of an autophagy modulator (e.g., ATG5 or ATG7) operatively linked to the promoter increases when the small molecule drug is administered to the subject or to the immune cells. In certain embodiments, the promoter may be operatively linked to gene engineering components (e.g., CRISPR/Cas9) configured to target and disrupt the autophagy modulator gene of the immune cell, such that administration of a small molecule drug to the cell is effective to stop ectopic expression of the autophagy modulator gene.

Further, the recombinant expression vectors can be made to include a suicide gene. As used herein, the term “suicide gene” refers to a gene that causes the cell expressing the suicide gene to die. The suicide gene can be a gene that confers sensitivity to an agent, e.g., a drug, upon the cell in which the gene is expressed, and causes the cell to die when the cell is contacted with or exposed to the agent. Suicide genes are known in the art and include, for example, the Herpes Simplex Virus (HSV) thymidine kinase (TK) gene, cytosine deaminase, purine nucleoside phosphorylase, and nitroreductase.

Included in the scope of the invention are conjugates, e.g., bioconjugates, comprising any of the autophagy modulators, CARs, polypeptides, or proteins (including any of the functional portions or variants thereof), host cells, nucleic acids, recombinant expression vectors, populations of host cells, or antibodies, or antigen binding portions thereof. Conjugates, as well as methods of synthesizing conjugates in general, are known in the art (See, for instance, Hudecz, F., Methods Mol. Biol. 298: 209-223 (2005) and Kirin et al., Inorg Chem. 44(15): 5405-5415 (2005)).

Also provided by the present disclosure is a nucleic acid comprising a nucleotide sequence encoding any of the autophagy modulators, CARs, polypeptides, or proteins described herein (including functional portions and functional variants thereof). Increased expression of autophagy modulators (e.g., ATG5 and ATG7) can increase the rate of autophagy in an immune cell. Increased autophagy can improve survival of the immune cell. Increased autophagy can also improve the ability of the immune cell to proliferate in response to stimulus. Further, increased autophagy can reduce exhaustion of the immune cell (e.g., T cell exhaustion and NK cell exhaustion).

In certain embodiments, a single vector expresses (i) a CAR and (ii) an autophagy modulator (e.g., either ATG5 or ATG7). In such vector, the autophagy modulator coding sequence may be upstream of the CAR leader sequence, or downstream of the CAR CD3C domain sequence. In some embodiments, the autophagy modulator is ATG5. In some embodiments, the autophagy modulator is ATG7. An IRES or a 2A peptide coding sequence is intercalated between the CAR and the autophagy modulator (e.g., either of ATG5 or ATG7). In some embodiments, the single vector expresses (i) a CAR, (ii) ATG5, and (iii) ATG7. An IRES or a 2A peptide is intercalated between each of the CAR, ATG5 and ATG7.

In one aspect, the disclosure provides a CAR, comprising an extracellular antigen-binding domain, a transmembrane domain and an intracellular signaling domain, wherein the extracellular antigen-binding domain binds the BCMA antigen. Various CAR comprising antigen-binding domains that bind to the BCMA antigen may be used, including those described in U.S. Pat. Nos. 9,765,342 and 10,294,304, U.S. Patent Publication Nos. 2018/0085444 and 2018/0187149, and International Patent Publication Nos. WO2018/085690, WO2019/090003, 2019/108900, each of which is incorporated by reference herein in its entirety.

In one embodiment, the autophagy modulator comprises an amino acid sequence having at least 50, at least 55, at least 60, at least 65, at least 70, at least 75, at least 80, at least 85, at least 90, at least 95, at least 98 or at least 99%, sequence identity with SEQ ID NO: 1.

In one embodiment, the autophagy modulator comprises an amino acid sequence having at least 50, at least 55, at least 60, at least 65, at least 70, at least 75, at least 80, at least 85, at least 90, at least 95, at least 98 or at least 99%, sequence identity with SEQ ID NO: 2.

In one embodiment, the autophagy modulator comprises an amino acid sequence having at least 50, at least 55, at least 60, at least 65, at least 70, at least 75, at least 80, at least 85, at least 90, at least 95, at least 98 or at least 99%, sequence identity with SEQ ID NO: 3.

In one embodiment, the autophagy modulator comprises an amino acid sequence having at least 50, at least 55, at least 60, at least 65, at least 70, at least 75, at least 80, at least 85, at least 90, at least 95, at least 98 or at least 99%, sequence identity with SEQ ID NO: 4.

In one aspect, the present disclosure provides isolated immunoresponsive cells comprising the CARs described herein, as well as one or more autophagy modulators described herein. In some embodiments, the isolated immunoresponsive cell is transduced with the CAR, for example, the CAR is constitutively expressed on the surface of the immunoresponsive cell. In some embodiments, the isolated immunoresponsive cell is transduced with the autophagy modulator, e.g., ATG5 or ATG7. In various embodiments, the immunoresponsive cell is transduced with the CAR and autophagy modulator coding sequences on separate vectors, e.g., lentiviral vectors. In various embodiments, the immunoresponsive cell is transduced with the CAR and the autophagy modulator on the same vector, e.g., the same lentiviral vector, with IRES or other such sequences allowing for transcription of both the CAR and the autophagy modulator from the same vector.

In one embodiment, the lentiviral vector comprising sequence encoding for an autophagy modulator comprises a polynucleotide sequence having at least 50, at least 55, at least 60, at least 65, at least 70, at least 75, at least 80, at least 85, at least 90, at least 95, at least 98 or at least 99%, sequence identity with SEQ ID NO: 5.

In one embodiment, the lentiviral vector comprising sequence encoding for an autophagy modulator comprises a polynucleotide sequence having at least 50, at least 55, at least 60, at least 65, at least 70, at least 75, at least 80, at least 85, at least 90, at least 95, at least 98 or at least 99%, sequence identity with SEQ ID NO: 6.

In one embodiment, the lentiviral vector comprising sequence encoding for an autophagy modulator comprises a polynucleotide sequence having at least 50, at least 55, at least 60, at least 65, at least 70, at least 75, at least 80, at least 85, at least 90, at least 95, at least 98 or at least 99%, sequence identity with SEQ ID NO: 7.

In one embodiment, the lentiviral vector comprising sequence encoding for an autophagy modulator comprises a polynucleotide sequence having at least 50, at least 55, at least 60, at least 65, at least 70, at least 75, at least 80, at least 85, at least 90, at least 95, at least 98 or at least 99%, sequence identity with SEQ ID NO: 8.

In certain embodiments, the isolated immunoresponsive cell is further transduced with at least one co-stimulatory ligand such that the immunoresponsive cell expresses the at least one co-stimulatory ligand. In certain embodiments, the at least one co-stimulatory ligand is selected from the group consisting of 4-1BBL, CD48, CD70, CD80, CD86, OX40L, TNFRSF14, and combinations thereof. In certain embodiments, the isolated immunoresponsive cell is further transduced with at least one cytokine such that the immunoresponsive cell secretes the at least one cytokine. In certain embodiments, the at least cytokine is selected from the group consisting of IL-2, IL-3, IL-6, IL-7, IL-11, IL-12, IL-15, IL-17, IL-21, and combinations thereof. In some embodiments, the isolated immunoresponsive cell is selected from the group consisting of a T lymphocyte (T cell), a Natural Killer (NK) cell, a cytotoxic T lymphocyte (CTL), a regulatory T cell, a human embryonic stem cell, a lymphoid progenitor cell, a T cell-precursor cell, and a pluripotent stem cell from which lymphoid cells may be differentiated.

In one embodiment, the CAR T cells of the disclosure can be generated by introducing a lentiviral vector comprising a desired CAR, for example, a CAR comprising anti-hK2, CD8a hinge and transmembrane domain, and human 4-1BB and CD3-zeta signaling domains, into the cells. The CAR T cells of the invention are able to replicate in vivo resulting in long-term persistence that can lead to sustained tumor control.

Embodiments of the invention further provide host cells comprising any of the recombinant expression vectors described herein. As used herein, the term “host cell” refers to any type of cell that can contain the recombinant expression vector. The host cell can be a eukaryotic cell, e.g., plant, animal, or algae, fungi, or can be a prokaryotic cell, e.g., bacteria or protozoa. The host cell can be a cultured cell or a primary cell, i.e., isolated directly from an organism, e.g., a human. The host cell can be an adherent cell or a suspended cell, i.e., a cell that grows in suspension. Suitable host cells are known in the art and include, for instance, DH5α E. coli cells, Chinese hamster ovarian cells, monkey VERO cells, COS cells, HEK293 cells, and the like. For purposes of amplifying or replicating the recombinant expression vector, the host cell may be a prokaryotic cell, e.g., a DH5a cell.

For purposes of producing a recombinant CAR, polypeptide, or protein, the host cell may be a mammalian cell. The host cell may be a human cell. While the host cell can be of any cell type, can originate from any type of tissue, and can be of any developmental stage, the host cell may be a peripheral blood lymphocyte (PBL). The host cell may be an immunoresponsive cell, such as a T cell or an NK cell. The host cell may comprise a single vector that encodes for both the recombinant CAR and the autophagy modulators. The host cell may comprise a single vector that encodes the recombinant CAR and a single vector that encodes autophagy modulators. In various embodiments, increased expression of one or more autophagy modulators (e.g., ATG5 and ATG7) can increase the rate of autophagy in the host cell. Increased autophagy in an immune cell as host cell can improve survival of the immune cell. Increased autophagy can also improve the ability of the immune cell to proliferate in response to stimulus. Further, increased autophagy can reduce exhaustion of the immune cell (e.g., T cell exhaustion and NK cell exhaustion).

In various embodiments, the genome of the host cell may be modified so as to increase transcription of, and/or expression of, autophagy modulators. The endogenous promoter of an autophagy modulator may be replaced by a stronger constitutive promoter. One or more endogenous enhancer elements of an autophagy modulator may be replaced by a stronger enhancer element. One or more additional copies of the autophagy modulator gene, that may or may not include various constitutive promoter and/or enhancer elements, may be introduced into the cell.

Various gene-editing and genome engineering technologies may be used to introduce autophagy modulator genes, constitutive promoters, and/or constitute enhancers into the host cell genome, such as a CRISPR/Cas9 system, a CRISPR/Cpf1 system a zinc finger nuclease system, a TALEN system, a meganuclease system, and argonauts. These systems are described in U.S. Patent Publication No. 2018/0258149, incorporated herein by reference in its entirety. An immune cell whose genome has been modified so as to increase the expression of an autophagy modulator (e.g., ATG5 or ATG7), and progeny of such immune cell, can have one or more of the following properties: increased survival, increased proliferation in response to antigen-based stimulus, and reduced propensity to undergo immune cell exhaustion.

In one aspect, the immune cells (e.g., CAR T cells and CAR NK cells) comprise an autophagy modulator gene (e.g., ATG5 or ATG7). In some embodiments, the immune cells further comprise a gene editing system targeted to any location on the genome. In some embodiments, the immune cells further comprise a gene editing system targeted to one or more sites within the autophagy modulator gene (including promoter and enhancer sequences). In some embodiments, the immune cells further comprise a gene editing system targeted to any location on the genome. The gene editing system may comprise a nucleic acid encoding one or more components of the gene editing system. In various embodiments, the gene editing system is selected from the group consisting of: a CRISPR/Cas9 system, CRISPR/Cpf1 system, a zinc finger nuclease system, a TALEN system, a meganuclease system, and an argonaut system.

In some embodiments, the gene editing system targets a promoter sequence upstream of the autophagy modulator gene (e.g., ATG5 or ATG7). The gene editing system may comprise sequence configured to replace the endogenous promoter sequence of the autophagy modulator gene with a constitutive promoter. Exemplary constitutive promoters include, but are not limited to, a TRAC promoter, a β-2m promoter, a CMV promoter, or an EF1a promoter. The gene editing system may be configured so as to insert the autophagy modulator gene into the TRAC locus, or into the β-2m locus. In some embodiments, the gene editing system may be configured so as to insert both the CAR and the autophagy modulator genes into the TRAC locus, or into the β-2m locus. To express the autophagy modulator concomitantly with the CAR, an IRES sequence or a 2A peptide sequence is intercalated between the autophagy modulator coding sequence and the CAR coding sequence. In certain embodiments, multiple autophagy modulators (e.g., both ATG5 and ATG7) can be expressed with the gene editing sequence, with an IRES sequence or a 2A peptide sequence intercalated between the autophagy modulator coding sequences.

In some embodiments, the gene editing system targets a sequence of the autophagy modulator gene so as to reduce expression of the autophagy modulator gene, where the autophagy modulator gene encodes a protein that inhibits autophagy (i.e., an autophagy inhibitor). The gene editing system can disrupt the expression of the autophagy modulator gene by introducing mutations into the coding sequence (such as by introducing a premature stop codon or a deletion), and/or by deleting all of, or a portion of, the promoter sequence. Exemplary autophagy inhibitors whose genes may be disrupted include, but are not limited to, G9a, mTOR, GADD45A, p38 MAPK, and SGK1.

In various embodiments, expression of autophagy inhibitor genes in the immune cell are silenced using RNA interference. Small interfering RNAs (siRNA) specific to autophagy inhibitors are introduced to the immune cell. Such siRNA consist of about approximately 20 bases of RNA sequence specific to an autophagy inhibitor, e.g., G9a, mTOR, GADD45A, p38 MAPK, and SGK1. In various embodiments, the immune cells are contacted with the siRNAs. The siRNAs may be effective to increase autophagy in the immune cell.

In various embodiments, the immune cells are contacted with micro RNAs (miRNAs) that are effective to activate autophagy. Exemplary miRNAs that can activate autophagy include, but are not limited to, miR-155 (Wang et al., PLOS Pathogens, 2013, 9(10): e1003697, miR-451 (Song et al., J. Cell. Mol. Med., 2014, 18(11), and miR-378 (Li et al., PNAS, 2018, 115 (46) E10849-E10858).

In various embodiments, the immune cells are engineered to overexpress an RNA that is effective to activate or increase the rate of autophagy. The overexpression and/or gene engineering may be conducted according to any of the methods described herein. Exemplary RNA sequences that can activate autophagy include, but are not limited to, HOTAIR (Yang et al., Molecular BioSystems, 2016, 12, 2605-2612), GBCDR1nc1 (Cai et al., Molecular Cancer, 2019, 18:82), and Malat1 (Si et al., Cellular & Molecular Biology Letters, 2019, 24:50).

In the various embodiments and aspects described herein, the T cell can be any T cell, such as a cultured T cell, e.g., a primary T cell, or a T cell from a cultured T cell line, e.g., Jurkat, SupT1, etc., or a T cell obtained from a mammal. If obtained from a mammal, the T cell can be obtained from numerous sources, including but not limited to bone marrow, blood, lymph node, the thymus, or other tissues or fluids. T cells can also be enriched for or purified. The T cell may be a human T cell. The T cell may be a T cell isolated from a human. The T cell can be any type of T cell and can be of any developmental stage, including but not limited to, CD4⁺/CD8⁺ double positive T cells, CD8⁺ T cells (e.g., cytotoxic T cells), CD4⁺ helper T cells, e.g., Th₁ and Th₂ cells, peripheral blood mononuclear cells (PBMCs), peripheral blood leukocytes (PBLs), tumor infiltrating cells, memory T cells, naïve T cells, and the like. The T cell may be a CD8⁺ T cell or a CD4⁺ T cell.

Without wishing to be bound by theory, the introduction of autophagy modulators into the T cell or NK cell may enhance proliferation, function and/or survival of the cell. This benefit can be helpful when T cells or NK cells are isolated from already-ill patients for use in CAR-T or CAR-NK therapy. Introduction of autophagy modulators into the T cell or NK cell may improve regulation of effector/memory differentiation. Introduction of autophagy modulators into the T cell or NK cell may reverse of T cell or NK cell dysfunction and exhaustion. One or more of these effects may thereby increase lymphocyte proliferation and/or function.

Without wishing to be bound by theory, the introduction of autophagy modulators into the T cell or NK cell may enhance mitochondrial function of the cell. This can be beneficial because mitochondria are responsible for the supply of energy to maintain cellular physiology and energy metabolism. Autophagy controls mitochondrial number and health, while at the same time mitochondria can influence the autophagic process. The cross-talk between these two systems could potentiate the contribution of both systems, thereby enhancing the cell's proliferative capacity while retaining more central memory and resulting in less exhaustion. One or more of these effects may thereby increase lymphocyte survival, proliferation, and/or function.

Without wishing to be bound by theory, the introduction of autophagy modulators into the T cell or NK cell may result in the generation of longer lasting and/or less differentiated cells. This can be beneficial because T cell or NK cell differentiation may impair adoptive immunotherapy and reduce efficacy. T cell differentiation markers include, but are not limited to, CD45 RA or RO, CD62L, CCR7, CD27, and CD28. NK cell differentiation markers include, but are not limited to, CD16, CD56, CD57, CD94, CD122, NKp30, NKG2D and KIR. One or more of these effects may thereby increase lymphocyte survival, proliferation, and/or function.

Engineered B Cells

In another aspect is provided a method for improving the function, survival, and/or effectiveness of a gene-engineered B cell, the method comprising contacting the immune cell with an autophagy modulator and/or a vector comprising a nucleotide sequence encoding the autophagy modulator. Without wishing to be bound by theory, autophagy plays important roles in B cell development, activation, and differentiation to accommodate the phenotypic and environmental changes encountered over the lifetime of the cell. Increased autophagy may improve the ability of a B cell to undergo such development, activation, and/or differentiation. The gene-engineered B cells may be used express a certain protein so as to treat various diseases and conditions where the body is unable to make that certain protein. The gene-engineered B cells may express an introduced protein, and be used to treat immune disorders where the introduced protein in the engineered B cells could be used to turn off abnormal immune responses, or to disarm infectious diseases by secreting known protective antibodies express a certain protein so as to treat various diseases and conditions where the body is unable to make that certain protein. Expression of an autophagy modulator in any of these engineered B cells may be effective to improve expression of the protein.

Pharmaceutical Compositions/Administration

In embodiments of the present disclosure, the CAR- and autophagy-modulator-expressing cells may be provided in compositions, e.g., suitable pharmaceutical composition(s) comprising the (i) CAR- and autophagy-modulator-expressing cells and (ii) a pharmaceutically acceptable carrier. In one aspect, the present disclosure provides pharmaceutical compositions comprising an effective amount of (i) a lymphocyte expressing one or more of the CARs and autophagy modulators described herein, and (ii) a pharmaceutically acceptable excipient. Pharmaceutical compositions of the present disclosure may comprise a CAR-expressing cell that also expresses an autophagy modulator, e.g., a plurality of CAR-expressing cells expressing autophagy modulators, as described herein, in combination with one or more pharmaceutically or physiologically acceptable carriers, excipients or diluents. A pharmaceutically acceptable carrier can be an ingredient in a pharmaceutical composition, other than an active ingredient, which is nontoxic to the subject.

In embodiments of the present disclosure, the tumor infiltrating lymphocytes that express one or more autophagy modulators may be provided in compositions, e.g., suitable pharmaceutical composition(s) comprising the (i) the tumor infiltrating lymphocytes that express one or more autophagy modulators and (ii) a pharmaceutically acceptable carrier. In one aspect, the present disclosure provides pharmaceutical compositions comprising an effective amount of (i) the tumor infiltrating lymphocytes that express one or more autophagy modulators described herein, and (ii) a pharmaceutically acceptable excipient. Pharmaceutical compositions of the present disclosure may comprise a tumor infiltrating lymphocytes that express one or more autophagy modulators, as described herein, in combination with one or more pharmaceutically or physiologically acceptable carriers, excipients or diluents. A pharmaceutically acceptable carrier can be an ingredient in a pharmaceutical composition, other than an active ingredient, which is nontoxic to the subject.

A pharmaceutically acceptable carrier can include, but is not limited to, a buffer, excipient, stabilizer, or preservative. Examples of pharmaceutically acceptable carriers are solvents, dispersion media, coatings, antibacterial and antifungal agents, isotonic and absorption delaying agents, and the like that are physiologically compatible, such as salts, buffers, antioxidants, saccharides, aqueous or non-aqueous carriers, preservatives, wetting agents, surfactants or emulsifying agents, or combinations thereof. The amounts of pharmaceutically acceptable carrier(s) in the pharmaceutical compositions may be determined experimentally based on the activities of the carrier(s) and the desired characteristics of the formulation, such as stability and/or minimal oxidation.

Such compositions may comprise buffers such as acetic acid, citric acid, formic acid, succinic acid, phosphoric acid, carbonic acid, malic acid, aspartic acid, histidine, boric acid, Tris buffers, HEPPSO, HEPES, neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); antibacterial and antifungal agents; and preservatives.

Compositions of the present disclosure can be formulated for a variety of means of parenteral or non-parenteral administration. In one embodiment, the compositions can be formulated for infusion or intravenous administration. Compositions disclosed herein can be provided, for example, as sterile liquid preparations, e.g., isotonic aqueous solutions, emulsions, suspensions, dispersions, or viscous compositions, which may be buffered to a desirable pH. Formulations suitable for oral administration can include liquid solutions, capsules, sachets, tablets, lozenges, and troches, powders liquid suspensions in an appropriate liquid and emulsions.

The term “pharmaceutically acceptable,” as used herein with regard to pharmaceutical compositions, means approved by a regulatory agency of the Federal or a state government or listed in the U.S. Pharmacopeia or other generally recognized pharmacopeia for use in animals and/or in humans.

In one aspect, the disclosure relates to administering a genetically modified T cell expressing an autophagy modulator and a CAR for the treatment of a subject having cancer or at risk of having cancer using lymphocyte infusion. In at least one embodiment, autologous lymphocyte infusion is used in the treatment. Autologous PBMCs are collected from a subject in need of treatment and T cells are activated and expanded using the methods described herein and known in the art and then infused back into the subject.

In another aspect, the disclosure relates to administering a tumor infiltrating lymphocyte expressing an autophagy modulator and a CAR for the treatment of a subject having cancer or at risk of having cancer using lymphocyte infusion.

In one aspect, the disclosure relates generally to the treatment of a subject at risk of developing cancer. The invention also includes treating a malignancy or an autoimmune disease in which chemotherapy and/or immunotherapy in a subject results in significant immunosuppression, thereby increasing the risk of the subject developing cancer. In one aspect, the present disclosure provides methods of preventing cancer, the methods comprising administering to a subject in need thereof an amount of a lymphocyte expressing one or more of the autophagy inhibitors described with one or more of the CARs described herein. In another aspect, the present disclosure provides methods of preventing cancer, the methods comprising administering to a subject in need thereof an amount of a tumor infiltrating lymphocyte expressing one or more of the autophagy inhibitors described herein.

In one aspect, the present disclosure provides methods of treating a subject having cancer, the methods comprising administering to a subject in need thereof a therapeutically effective amount of a lymphocyte expressing one or more of the autophagy inhibitors described herein with one or more of the CARs described herein, whereby the lymphocyte induces or modulates killing of cancer cells in the subject. In another aspect, the present disclosure provides methods of treating a subject having cancer, the methods comprising administering to a subject in need thereof a therapeutically effective amount of a tumor infiltrating lymphocyte expressing one or more of the autophagy inhibitors described herein, whereby the lymphocyte induces or modulates killing of cancer cells in the subject.

In another aspect, the present disclosure provides methods of reducing tumor burden in a subject having cancer, the methods comprising administering to a subject in need thereof a therapeutically effective amount of a lymphocyte expressing one or more of the autophagy inhibitors described with one or more of the CARs described herein, whereby the lymphocyte induces killing of cancer cells in the subject. In another aspect, the present disclosure provides methods of reducing tumor burden in a subject having cancer, the methods comprising administering to a subject in need thereof a therapeutically effective amount of a tumor infiltrating lymphocyte expressing one or more of the autophagy inhibitors described, whereby the lymphocyte induces killing of cancer cells in the subject.

In another aspect, the present disclosure provides methods of increasing survival of a subject having cancer, the methods comprising administering to a subject in need thereof a therapeutically effective amount of a lymphocyte expressing one or more of the autophagy inhibitors described herein with one or more of the CARs described, whereby the survival of the subject is lengthened. In another aspect, the present disclosure provides methods of increasing survival of a subject having cancer, the methods comprising administering to a subject in need thereof a therapeutically effective amount of a tumor infiltrating lymphocyte expressing one or more of the autophagy inhibitors described herein, whereby the survival of the subject is lengthened.

Generally, (i) the lymphocytes expressing the autophagy inhibitors and the CAR(s), and (ii) the tumor infiltrating lymphocytes expressing the autophagy inhibitors, induce killing of cancer cells in the subject and result in reduction or eradication of the tumors/cancer cells in the subject.

In one aspect, the methods described herein are applicable to treatment of noncancerous conditions that are at risk of developing into a cancerous condition.

In one aspect, a method of targeted killing of a cancer cell is disclosed, the method comprising contacting the cancer cell with a lymphocyte expressing one or more of the of the autophagy inhibitors described with one or more of CARs described, whereby the lymphocyte induces killing of the cancer cell. A non-limiting list of cancer cells, inclusive of metastatic cancer cells, that can be targeted include prostate cancer, and combinations thereof. In one embodiment, the cancer cell is a prostate cancer cell.

Pharmaceutical compositions of the present disclosure may be administered in a manner appropriate to the disease to be treated (or prevented). The quantity and frequency of administration will be determined by such factors as the condition of the subject, and the type and severity of the subject's disease, although appropriate dosages may be determined by clinical trials.

The terms “treat” or “treatment” refer to therapeutic treatment wherein the object is to slow down (lessen) an undesired physiological change or disease, or provide a beneficial or desired clinical outcome during treatment. Beneficial or desired clinical outcomes include alleviation of symptoms, diminishment of extent of disease, stabilized (i.e., not worsening) state of disease, delay or slowing of disease progression, amelioration or palliation of the disease state, and/or remission (whether partial or total), whether detectable or undetectable. “Treatment” can also mean prolonging survival as compared to expected survival if a subject was not receiving treatment. Those in need of treatment include those subjects already with the undesired physiological change or disease as well as those subjects prone to have the physiological change or disease.

A “therapeutically effective amount” or “effective amount”, used interchangeably herein, refers to an amount effective, at dosages and for periods of time necessary, to achieve a desired therapeutic result. A therapeutically effective amount may vary according to factors such as the disease state, age, sex, and weight of the individual, and the ability of a therapeutic or a combination of therapeutics to elicit a desired response in the individual. Example indicators of an effective therapeutic or combination of therapeutics that include, for example, improved wellbeing of the patient, reduction of a tumor burden, arrested or slowed growth of a tumor, and/or absence of metastasis of cancer cells to other locations in the body.

As used herein, the term “subject” refers to an animal. The terms “subject” and “patient” may be used interchangeably herein in reference to a subject. As such, a “subject” includes a human that is being treated for a disease, or prevention of a disease, as a patient. The methods described herein may be used to treat an animal subject belonging to any classification. Examples of such animals include mammals. Mammals, include, but are not limited to, mammals of the order Rodentia, such as mice and hamsters, and mammals of the order Logomorpha, such as rabbits. The mammals may be from the order Carnivora, including felines (cats) and canines (dogs). The mammals may be from the order Artiodactyla, including bovines (cows) and swines (pigs) or of the order Perssodactyla, including equines (horses). The mammals may be of the order Primates, Ceboids, or Simoids (monkeys) or of the order Anthropoids (humans and apes). In one embodiment, the mammal is a human.

When a therapeutically effective amount is indicated, the precise amount of the compositions of the present disclosure to be administered can be determined by a physician with consideration of individual differences in age, weight, tumor size, extent of infection or metastasis, and condition of the subject. It can generally be stated that a pharmaceutical composition comprising the T cells, NK cells or B cells described herein may be administered at a dosage of about 10⁴ to about 10¹⁰ cells/kg body weight, in some instances about 10⁵ to about 10⁶ cells/kg body weight, including all integer values within those ranges. In some embodiments, a pharmaceutical composition comprising the T cells, NK cells or B cells described herein may be administered at a dosage of about 10⁶ cells/kg body weight. T cell, NK cell or B cell compositions may also be administered multiple times at these dosages. The cells can be administered by using infusion techniques that are commonly known in immunotherapy (see, e.g., Rosenberg et al., New Eng. J. of Med. 319:1676, 1988).

Delivery systems useful in the context of embodiments of the invention may include time-released, delayed release, and sustained release delivery systems such that the delivery of the T cell, NK cell or B cell compositions occurs prior to, and with sufficient time to cause, sensitization of the site to be treated. The composition can be used in conjunction with other therapeutic agents or therapies. Such systems can avoid repeated administrations of the composition, thereby increasing convenience to the subject and the physician, and may be particularly suitable for certain composition embodiments of the invention.

Many types of release delivery systems are available and known to those of ordinary skill in the art. They include polymer base systems such as poly(lactide-glycolide), copolyoxalates, polyesteramides, polyorthoesters, polycaprolactones, polyhydroxybutyric acid, and polyanhydrides. Microcapsules of the foregoing polymers containing drugs are described in, for example, U.S. Pat. No. 5,075,109. Delivery systems also include non-polymer systems that are lipids including sterols such as cholesterol, cholesterol esters, and fatty acids or neutral fats such as mono-di- and tri-glycerides; sylastic systems; peptide based systems; hydrogel release systems; wax coatings; compressed tablets using conventional binders and excipients; partially fused implants; and the like. Specific examples include, but are not limited to: (a) erosional systems in which the active composition is contained in a form within a matrix such as those described in U.S. Pat. Nos. 4,452,775; 4,667,014; 4,748,034; and 5,239,660 and (b) diffusional systems in which an active component permeates at a controlled rate from a polymer such as described in U.S. Pat. Nos. 3,854,480 and 3,832,253. In addition, pump-based hardware delivery systems can be used, some of which are adapted for implantation.

In certain aspects, it may be desirable to administer activated T cells, NK cells or B cells to a subject and then subsequently redraw blood (or have an apheresis performed), activate the T cells, NK cells or B cells according to the present disclosure, and reinfuse the subject with these activated and expanded T cells, NK cells or B cells. This process can be carried out multiple times every few weeks. In certain aspects, T cells, NK cells or B cells can be activated from blood draws of from 10 cc to 400 cc. In certain aspects, T cells, NK cells or B cells are activated from blood draws of 20 cc, 30 cc, 40 cc, 50 cc, 60 cc, 70 cc, 80 cc, 90 cc, or 100 cc.

The administration of the CAR-T cells and compositions may be carried out in any manner, e.g., by parenteral or nonparenteral administration, including by aerosol inhalation, injection, infusions, ingestion, transfusion, implantation or transplantation. For example, the CAR-T cells and compositions described herein may be administered to a patient trans-arterially, intradermally, subcutaneously, intratumorally, intramedullary, intranodally, intramuscularly, by intravenous (i.v.) injection, or intraperitoneally. In one aspect, the compositions of the present disclosure are administered by i.v. injection. In one aspect, the compositions of the present disclosure are administered to a subject by intradermal or subcutaneous injection. The compositions of T cells or NK cells may be injected, for instance, directly into a tumor, lymph node, tissue, organ, or site of infection.

Administration can be autologous or non-autologous. For example, immunoresponsive cells expressing a human BCMA-specific CAR can be obtained from one subject, and administered to the same subject or a different, compatible subject. Peripheral blood derived T cells or NK cells of the present disclosure, or expanded T cells or NK cells (e.g., in vivo, ex vivo or in vitro derived) can be administered via, e.g., intravenous injection, localized injection, systemic injection, catheter administration, or parenteral administration.

In particular embodiments, subjects may undergo leukapheresis, wherein leukocytes are collected, enriched, or depleted ex vivo to select and/or isolate the cells of interest, e.g., T cells or NK cells. These T cell or NK cell isolates may be expanded by methods known in the art and treated such that one or more CAR constructs of the present disclosure may be introduced, thereby creating a CAR-T cell or a CAR-NK cell. Subjects in need thereof may subsequently undergo standard treatment with high dose chemotherapy followed by peripheral blood stem cell transplantation. In certain aspects, following or concurrent with the transplant, subjects receive an infusion of the expanded CAR-T cells or CAR-NK cells. In one aspect, expanded cells are administered before or following surgery.

The dosage administered to a patient having a malignancy is sufficient to alleviate or at least partially arrest the disease being treated (“therapeutically effective amount”). The dosage of the above treatments to be administered to a subject will vary with the precise nature of the condition being treated and the recipient of the treatment. The scaling of dosages for human administration can be performed according to practices generally accepted in the art.

The CAR T cells of the invention can undergo in vivo T cell expansion and can establish BCMA-specific memory cells that persist at high levels for an extended amount of time in blood and bone marrow. In some instances, the CAR T cells of the invention infused into a subject can eliminate cancer cells in vivo in subjects with advanced chemotherapy-resistant cancer.

In one embodiment, a CAR of the present disclosure is introduced into T cells, e.g., using in vitro transcription, and the subject (e.g., human) receives an initial administration of CAR-T cells of the disclosure, and one or more subsequent administrations of the CAR-T cells, wherein the one or more subsequent administrations are administered less than 15 days, e.g., 14, 13, 12, 11, 10, 9, 8, 7, 6, 5, 4, 3, or 2 days after the previous administration. In one embodiment, more than one administration of the CAR-T cells are administered to the subject (e.g., human) per week, e.g., 2, 3, or 4 administrations of the CAR-T cells are administered per week. In one embodiment, the subject receives more than one administration of the CAR-T cells per week (e.g., 2, 3 or 4 administrations per week) (also referred to herein as a cycle), followed by a week of no CAR-T cell administrations, and then one or more additional administration of the CAR-T cells (e.g., more than one administration of the CAR-T cells per week) is administered to the subject. In another embodiment, the subject receives more than one cycle of CAR-T cells, and the time between each cycle is less than 10, 9, 8, 7, 6, 5, 4, or 3 days. In one embodiment, the CAR-T cells are administered every other day for 3 administrations per week. In one embodiment, the CAR-T cells are administered for at least two, three, four, five, six, seven, eight or more weeks.

In one embodiment, a CAR of the present disclosure is introduced into NK cells, e.g., using in vitro transcription, and the subject (e.g., human) receives an initial administration of CAR-NK cells of the disclosure, and one or more subsequent administrations of the CAR-NK cells, wherein the one or more subsequent administrations are administered less than 15 days, e.g., 14, 13, 12, 11, 10, 9, 8, 7, 6, 5, 4, 3, or 2 days after the previous administration. In one embodiment, more than one administration of the CAR-NK cells are administered to the subject (e.g., human) per week, e.g., 2, 3, or 4 administrations of the CAR-NK cells are administered per week. In one embodiment, the subject receives more than one administration of the CAR-NK cells per week (e.g., 2, 3 or 4 administrations per week) (also referred to herein as a cycle), followed by a week of no CAR-NK cell administrations, and then one or more additional administration of the CAR-NK cells (e.g., more than one administration of the CAR-NK cells per week) is administered to the subject. In another embodiment, the subject receives more than one cycle of CAR-NK cells, and the time between each cycle is less than 10, 9, 8, 7, 6, 5, 4, or 3 days. In one embodiment, the CAR-NK cells are administered every other day for 3 administrations per week. In one embodiment, the CAR-NK cells are administered for at least two, three, four, five, six, seven, eight or more weeks.

In one embodiment, administration may be repeated after one day, two days, three days, four days, five days, six days, one week, two weeks, three weeks, one month, five weeks, six weeks, seven weeks, two months, three months, four months, five months, six months or longer. Repeated courses of treatment are also possible, as is chronic administration. The repeated administration may be at the same dose or at a different dose.

The CAR-T and CAR-NK cells may be administered in the methods of the invention by maintenance therapy, such as, e.g., once a week for a period of 6 months or more.

In one embodiment, CAR-T cells are generated using lentiviral viral vectors, such as lentivirus. CAR-T cells generated with such viral vectors will generally have stable CAR expression. In this embodiment, the CAR-T cells also comprise an autophagy modulator coding sequence on the same lentiviral viral vector, or on an additional lentiviral viral vector.

In one embodiment, CAR-NK cells are generated using lentiviral viral vectors, such as lentivirus. CAR-NK cells generated with such viral vectors will generally have stable CAR expression. In this embodiment, the CAR-NK cells also comprise an autophagy modulator coding sequence on the same lentiviral viral vector, or on an additional lentiviral viral vector.

In one embodiment, CAR-T or CAR-NK cells transiently express CAR vectors for 4, 5, 6, 7, 8, 9, 10, 11, 12, 13, 14, 15 days after transduction. Transient expression of CARs can be affected by RNA CAR vector delivery. The CARs may transiently express an autophagy modulator as well. Alternatively, the CARs may express an autophagy modulator in a viral vector. In one embodiment, the CAR RNA and/or the autophagy modulator vector are transduced into the T cell by electroporation. In another embodiment, in the CAR-T or CAR-NK cell, the promoter of an endogenous autophagy modulator gene is replaced with a constitutive promoter (e.g., a TRAC promoter, a β-2m promoter, a CMV promoter, or an EF1a promoter). Replacement of the promoter may be undertaken using a gene editing system (e.g., a CRISPR/Cas9 system, a CRISPR/Cpf1 system a zinc finger nuclease system, a TALEN system, or a meganuclease system).

If a patient is at high risk of generating an anti-CAR antibody response during the course of transient CAR therapy (such as those generated by RNA transductions), CAR-T or CAR-NK infusion breaks should not last more than ten to fourteen days.

A CAR-expressing cell described herein may be used in combination with other known agents and therapies. Administered “in combination”, as used herein, means that two (or more) different treatments are delivered to the subject during the course of the subject's treatment e.g., the two or more treatments are delivered after the subject has been diagnosed with the cancer and before the cancer has been cured or eliminated or treatment has ceased for other reasons. In some embodiments, the delivery of one treatment is still occurring when the delivery of the second begins, so that there is overlap in terms of administration. This is sometimes referred to herein as “simultaneous” or “concurrent delivery”. In other embodiments, the delivery of one treatment ends before the delivery of the other treatment begins. In some embodiments of either case, the treatment is more effective because of combined administration. For example, the second treatment is more effective, e.g., an equivalent effect is seen with less of the second treatment, or the second treatment reduces symptoms to a greater extent, than would be seen if the second treatment were administered in the absence of the first treatment, or the analogous situation is seen with the first treatment. In some embodiments, delivery is such that the reduction in a symptom, or other parameter related to the disorder is greater than what would be observed with one treatment delivered in the absence of the other. The effect of the two treatments can be partially additive, wholly additive, or greater than additive. The delivery can be such that an effect of the first treatment delivered is still detectable when the second is delivered.

In one embodiment, other therapeutic agents such as factors may be administered before, after, or at the same time (simultaneous with) as the CAR-T or CAR-NK cells, including, but not limited to, interleukins, e.g. IL-2, IL-3, IL 6, IL-7, IL-11, IL-12, IL-15, IL-21, as well as the other interleukins, colony stimulating factors, such as G-, M- and GM-CSF, and interferons, e.g., γ-interferon.

A CAR-expressing cell described herein and the at least one additional therapeutic agent can be administered simultaneously, in the same or in separate compositions, or sequentially. For sequential administration, the CAR-expressing cell described herein can be administered first, and the additional agent can be administered second, or the order of administration can be reversed.

In further embodiments, a CAR-expressing cell described herein may be used in a treatment regimen in combination with surgery, radiation, chemotherapy, immunosuppressive agents, such as methotrexate, cyclosporin, azathioprine, mycophenolate, and FK506, antibodies, or other immunoablative agents such as anti-CD3 antibodies or other antibody therapies, cytoxin, fludarabine, cyclosporin, FK506, rapamycin, mycophenolic acid, steroids, FR901228, cytokines, and irradiation.

In one embodiment, a CAR-expressing cell described herein can be used in combination with a chemotherapeutic agent. Example chemotherapeutic agents include, but are not limited to, an anthracycline (e.g., doxorubicin (e.g., liposomal doxorubicin)), a vinca alkaloid (e.g., vinblastine, vincristine, vindesine, vinorelbine), an alkylating agent (e.g., cyclophosphamide, decarbazine, melphalan, ifosfamide, temozolomide), an immune cell antibody (e.g., alemtuzamab, gemtuzumab, rituximab, tositumomab), an antimetabolite (including, e.g., folic acid antagonists, pyrimidine analogs, purine analogs and adenosine deaminase inhibitors (e.g., fludarabine)), an mTOR inhibitor, a TNFR glucocorticoid induced TNFR related protein (GITR) agonist, a proteasome inhibitor (e.g., aclacinomycin A, gliotoxin or bortezomib), an immunomodulator such as thalidomide or a thalidomide derivative (e.g., lenalidomide).

A non-exhaustive list of chemotherapeutic agents considered for use in combination therapies include anastrozole (Arimidex®), bicalutamide (Casodex®), bleomycin sulfate (Blenoxane®), busulfan (Myleran®), leucovorin calcium, melphalan (Alkeran®), 6-mercaptopurine (Purinethol®), methotrexate (Folex®), mitoxantrone (Novantrone®), mylotarg, paclitaxel (Taxol®), phoenix (Yttrium90/MX-DTPA), pentostatin, polifeprosan 20 with carmustine implant (Gliadel®), dactinomycin (Actinomycin D, Cosmegan), daunorubicin hydrochloride (Cerubidine®), daunorubicin citrate liposome injection (DaunoXome®), dexamethasone, docetaxel (Taxotere®), doxorubicin hydrochloride (Adriamycin®, Rubex®), etoposide (Vepesid®), busulfan injection (Busulfex®), capecitabine (Xeloda®), N4-pentoxycarbonyl-5-deoxy-5-fluorocytidine, carboplatin (Paraplatin®), carmustine (BiCNU®), chlorambucil (Leukeran®), cisplatin (Platinol®), cladribine (Leustatin®), cyclophosphamide (Cytoxan® or Neosar®), cytarabine, cytosine arabinoside (Cytosar-U®), cytarabine liposome injection (DepoCyt®), dacarbazine (DTIC-Dome®), fludarabine phosphate (Fludara®), 5-fluorouracil (Adrucil®, Efudex®), flutamide (Eulexin®), tezacitibine, Gemcitabine (difluorodeoxycitidine), hydroxyurea (Hydrea®), Idarubicin (Idamycin®), ifosfamide (IFEX®), irinotecan (Camptosar®), L-asparaginase (ELSPAR®), tamoxifen citrate (Nolvadex®), teniposide (Vumon®), 6-thioguanine, thiotepa, tirapazamine (Tirazone®), topotecan hydrochloride for injection (Hycamptin®), vinblastine (Velban®), vincristine (Oncovin®), and vinorelbine (Navelbine®).

Example alkylating agents include, without limitation, nitrogen mustards, ethylenimine derivatives, alkyl sulfonates, nitrosoureas and triazenes): uracil mustard (Aminouracil Mustard®, Chlorethaminacil®, Haemanthamine®, Nordopan®, Uracil Nitrogen Mustard®, Uracillost®, Uracilmostaza®, Uramustin®, Uramustine®), chlormethine (Mustargen®), cyclophosphamide (Cytoxan®, Neosar®, Clafen®, Endoxan®, Procytox®, Revimmune™), ifosfamide (Mitoxana®), melphalan (Alkeran®), Chlorambucil (Leukeran®), pipobroman (Amedel®, Vercyte®), triethylenemelamine (Hemel®, Hexylen®, Hexastat®), Demethyldopan®, Desmethyldopan®, triethylenethiophosphoramine, Temozolomide (Temodar®), thiotepa (Thioplex®), busulfan (Busilvex®, Myleran®), carmustine (BiCNU®), lomustine (CeeNU®), streptozocin (Zanosar®), and Dacarbazine (DTIC-Dome®). Additional example alkylating agents include, without limitation, Oxaliplatin (Eloxatin®); Melphalan (also known as L-PAM, L-sarcolysin, and phenylalanine mustard, Alkeran®); Altretamine (also known as hexamethylmelamine (HMM), Hexylen®); Carmustine (BiCNU®); Bendamustine (Treanda®); Busulfan (Busulfex® and Myleran®); Carboplatin (Paraplatin®); Temozolomide (Temodar® and Temodal®); Dactinomycin (also known as actinomycin-D, Cosmegen®); Lomustine (also known as CCNU, CeeNU®); Cisplatin (also known as CDDP, Platinol® and Platinol®-AQ); Chlorambucil (Leukeran®); Cyclophosphamide (Cytoxan® and Neosar®); Dacarbazine (also known as DTIC, DIC and imidazole carboxamide, DTIC-Dome®); Altretamine (also known as hexamethylmelamine (HMM), Hexylen®); Ifosfamide (Ifex®); Prednumustine; Procarbazine (Matulane®); Mechlorethamine (also known as nitrogen mustard, mustine and mechloroethamine hydrochloride, Mustargen®); Streptozocin (Zanosar®); Thiotepa (also known as thiophosphoamide, TESPA and TSPA, Thioplex®); Cyclophosphamide (Endoxan®, Cytoxan®, Neosar®, Procytox®, Revimmune®); and Bendamustine HCl (Treanda®).

Examples of immunomodulators useful herein include, but are not limited to, e.g., afutuzumab (available from Roche®); pegfilgrastim (Neulasta®); lenalidomide (CC-5013, Revlimid®); thalidomide (Thalomid®), actimid (CC4047); and IRX-2 (mixture of human cytokines including interleukin 1, interleukin 2, and interferon-γ, CAS 951209-71-5, available from IRX Therapeutics).

In one embodiment, the subject can be administered an agent which enhances the activity of a CAR-expressing cell. For example, in one embodiment, the agent can be an agent which inhibits an inhibitory molecule. Inhibitory molecules, e.g., Programmed Death 1 (PD1), can, in some embodiments, decrease the ability of a CAR-expressing cell to mount an immune effector response. Examples of inhibitory molecules include PD1, PD-L1, CTLA4, TIM3, LAG3, VISTA, BTLA, TIGIT, LAIR1, CD160, 2B4 and TGFR beta.

A description of example embodiments follows.

1. An immune cell expressing a first vector comprising a nucleotide sequence encoding a chimeric antigen receptor (CAR) and a second vector comprising a nucleotide sequence encoding an autophagy modulator.

2. An immune cell expressing a vector comprising a first nucleotide sequence encoding a chimeric antigen receptor (CAR) and a second nucleotide sequence encoding an autophagy modulator.

3. An immune cell comprising a vector comprising a nucleotide sequence encoding an autophagy modulator.

4. The immune cell of embodiment 3, wherein the immune cell further comprises a CAR.

5. The immune cell of any one of embodiments 1, 2 and 4, wherein the genome of the immune cell comprises one or more additional autophagy modulator genes.

6. The immune cell of any one of embodiments 1, 2 and 4, wherein a promoter of an autophagy modulator gene is replaced with a constitutive promoter.

7. The immune cell of any one of embodiments 1, 2, 4 and 6, wherein an enhancer sequence of an autophagy modulator gene is replaced with a second enhancer sequence that is effective to increase transcription of the autophagy modulator gene.

8. The immune cell of any one of embodiments 1 to 7, wherein the immune cell is a lymphocyte.

9. The immune cell of embodiment 8, wherein the immune cell is a tumor penetrating lymphocyte.

10. The immune cell of embodiment 8 or embodiment 9, wherein the immune cell is a T cell, a Natural Killer (NK) cell, or a B cell.

11. The immune cell of any one of embodiments 1, 2 and 3 to 9, wherein the CAR comprises an extracellular domain that specifically binds to the B-cell maturation antigen (BCMA), a CD19 antigen, a CD30 antigen, a CD123 antigen, an FLT3 antigen, and kallikrein-2 antigen.

12. The immune cell of any one of embodiments 1 to 11, wherein the autophagy modulator is ATG1, ATG2, ATG3, ATG4, ATG5, ATG6, ATG7, ATG8, ATG8, ATG10, ATG11, ATG12, ATG13, ATG14, ATG15, ATG16, ATG17, ATG18, ATG19, ATG20, ATG21, ATG22, ATG23, ATG24, ATG25, ATG26, ATG27, ATG28, ATG29, ATG30, ATG31, ATG101, LC3, RAB7, VPS15, VPS34, VPS35, LC3I, LC3II, UVRAG, Beclin1, Protor, CAMKKbeta, BCL2, BCL-XL, AKT, ULK1, ULK2, ULK3, ULK4, DapK1, FIP200, TSC1, TSC2, STRAD, AMPK, Redd1, LKB, M025, PTEN, mTOR, Deptor, Rictor, Protor, PRAS40, LST8, Rheb, RAG A, RAG B, RAG C, RAG D, Raptor, PDK1, PI3K, IRS1, Insulin/IGF1 receptor, ERK, Rab40b, p53, DRAM1, NDFIP, MEK, RAF, SIN1, MAP4K3, Plac8, Dominant negative (DN) Rab7, Rab7, Dominant active (DA) Rab7, SLC7A5, or SLC3A2.

13. The immune cell of embodiment 12, wherein the autophagy modulator is ATG5.

14. The immune cell of embodiment 13, wherein the ATG5 comprises an amino acid sequence at least 95% identical to the sequence of MTDDKDVLRDVWFGRIPTCFTLYQDEITEREAEPYYLLLPRVSYLTLVTDKVKKH FQKVMRQEDISEIWFEYEGTPLKWHYPIGLLFDLLASSSALPWNITVHFKSFPEKDL LHCPSKDAIEAHFMSCMKEADALKHKSQVINEMQKKDHKQLWMGLQNDRFDQF WAINRKLMEYPAEENGFRYIPFRIYQTTTERPFIQKLFRPVAADGQLHTLGDLLKE VCPSAIDPEDGEKKNQVMIHGIEPMLETPLQWLSEHLSYPDNFLHISIIPQPTD (SEQ ID NO: 1).

15. The immune cell of embodiment 13, wherein the ATG5 comprises an amino acid sequence at least 95% identical to the sequence of MTDDKDVLRDVWFGRIPTCFTLYQDEITEREAEPYYLLLPRVSYLTLVTDKVKKH FQKVMRQEDVSEIWFEYEGTPLKWHYPIGLLFDLLASSSALPWNITVHFKSFPEKD LLHCPSKDAVEAHFMSCMKEADALKHKSQVINEMQKKDHKQLWMGLQNDRFD QFWAINRKLMEYPPEENGFRYIPFRIYQTTTERPFIQKLFRPVAADGQLHTLGDLLR EVCPSAVAPEDGEKRSQVMIHGIEPMLETPLQWLSEHLSYPDNFLHISIVPQPTD (SEQ ID NO: 2).

16. The immune cell of embodiment 12, wherein the autophagy modulator is ATG7.

17. The immune cell of embodiment 16, wherein the ATG7 comprises an amino acid sequence at least 95% identical to the sequence of MAAATGDPGLSKLQFAPFSSALDVGFWHELTQKKLNEYRLDEAPKDIKGYYYNG DSAGLPARLTLEFSAFDMSAPTPARCCPAIGTLYNTNTLESFKTADKKLLLEQAAN EIWESIKSGTALENPVLLNKFLLLTFADLKKYHFYYWFCYPALCLPESLPLIQGPVG LDQRFSLKQIEALECAYDNLCQTEGVTALPYFLIKYDENMVLVSLLKHYSDFFQG QRTKITIGVYDPCNLAQYPGWPLRNFLVLAAHRWSSSFQSVEVVCFRDRTMQGA RDVAHSIIFEVKLPEMAFSPDCPKAVGWEKNQKGGMGPRMVNLSECMDPKRLAE SSVDLNLKLMCWRLVPTLDLDKVVSVKCLLLGAGTLGCNVARTLMGWGVRHIT FVDNAKISYSNPVRQPLYEFEDCLGGGKPKALAAADRLQKIFPGVNARGFNMSIP MPGHPVNFSSVTLEQARRDVEQLEQLIESHDVVFLLMDTRESRWLPAVIAASKRK LVINAALGFDTFVVMRHGLKKPKQQGAGDLCPNHPVASADLLGSSLFANIPGYKL GCYFCNDVVAPGDSTRDRTLDQQCTVSRPGLAVIAGALAVELMVSVLQHPEGGY AIASSSDDRMNEPPTSLGLVPHQIRGFLSRFDNVLPVSLAFDKCTACSSKVLDQYE REGFNFLAKVFNSSHSFLEDLTGLTLLHQETQAAEIWDMSDDETI (SEQ ID NO: 3).

18. The immune cell of embodiment 16, wherein the ATG7 comprises an amino acid sequence at least 95% identical to the sequence of MGDPGLAKLQFAPFNSALDVGFWHELTQKKLNEYRLDEAPKDIKGYYYNGDSAG LPTRLTLEFSAFDMSASTPAHCCPAMGTLHNTNTLEAFKTADKKLLLEQSANEIW EAIKSGAALENPMLLNKFLLLTFADLKKYHFYYWFCCPALCLPESIPLIRGPVSLDQ RLSPKQIQALEHAYDDLCRAEGVTALPYFLFKYDDDTVLVSLLKHYSDFFQGQRT KITVGVYDPCNLAQYPGWPLRNFLVLAAHRWSGSFQSVEVLCFRDRTMQGARDV THSIIFEVKLPEMAFSPDCPKAVGWEKNQKGGMGPRMVNLSGCMDPKRLAESSV DLNLKLMCWRLVPTLDLDKVVSVKCLLLGAGTLGCNVARTLMGWGVRHVTFV DNAKISYSNPVRQPLYEFEDCLGGGKPKALAAAERLQKIFPGVNARGFNMSIPMP GHPVNFSDVTMEQARRDVEQLEQLIDNHDVIFLLMDTRESRWLPTVIAASKRKLV INAALGFDTFVVMRHGLKKPKQQGAGDLCPSHLVAPADLGSSLFANIPGYKLGCY FCNDVVAPGDSTRDRTLDQQCTVSRPGLAVIAGALAVELMVSVLQHPEGGYAIAS SSDDRMNEPPTSLGLVPHQIRGFLSRFDNVLPVSLAFDKCTACSPKVLDQYEREGF TFLAKVFNSSHSFLEDLTGLTLLHQETQAAEIWDMSDEETV (SEQ ID NO: 4).

19. The immune cell of any one of embodiments 1 to 18, wherein the vector is a lentiviral vector.

20. The immune cell of any one of embodiments 1 to 19, wherein the expression of the autophagy modulator is at least four times the level of expression of the autophagy modulator in a comparable immune cell with normal expression of the autophagy inhibitor.

21. The immune cell of any one of embodiments 1 to 20, wherein the cytotoxic activity of the immune cell is not lower than that of a comparable immune cell with normal expression of the autophagy inhibitor.

22. The immune cell of any one of embodiments 1 to 21, wherein the immune cell is able to proliferate to a greater extent than a comparable immune cell with normal expression of the autophagy inhibitor.

23. The immune cell of any one of embodiments 1 to 21, wherein the immune cell enters a state of T cell exhaustion at a later time than a comparable immune cell with normal expression of the autophagy inhibitor.

24. The immune cell of any one of embodiments 1 to 21, wherein the immune cell does not undergo T cell exhaustion.

25. A pharmaceutical composition comprising an effective amount of the immune cell of any one of embodiments 1 to 24 and a pharmaceutically acceptable excipient.

26. A method of preparing the immune cells of any one of embodiments 1 to 24, the method comprising introducing the first and second vectors into an immune cell.

27. The method of embodiment 26, wherein the first vector, the second vector, or both the first and second vectors are transduced into the immune cell.

28. The method of embodiment 27, wherein the first vector is a viral vector, the second vector is a viral vector, or both the first vector and the second vector are viral vectors.

29. The method of embodiment 26, wherein a gene editing system is used to introduce the first vector and/or the second vector into the immune cell.

30. The method of embodiment 29, wherein the gene editing system is selected from the group consisting of a CRISPR/Cas9 system, a CRISPR/Cpf1 system a zinc finger nuclease system, a TALEN system, and a meganuclease system.

31. The method of embodiment 29 or embodiment 30, wherein the first vector and/or the second vector is integrated into the genome of the immune cell.

32. The method of embodiment 31, wherein the first vector and/or the second vector is integrated into a TRAC locus of the genome.

33. A method of preparing the immune cells of any one of embodiments 2 to 24, the method comprising introducing the vector into an immune cell.

34. The method of embodiment 33, wherein the vector is transduced into the immune cell.

35. The method of embodiment 33 or embodiment 34, wherein the vector is a viral vector.

36. The method of embodiment 33, wherein a gene editing system is used to introduce the vector into the immune cell.

37. The method of embodiment 36, wherein the gene editing system is selected from the group consisting of a CRISPR/Cas9 system, a CRISPR/Cpf1 system a zinc finger nuclease system, a TALEN system, and a meganuclease system.

38. The method of embodiment 36 or embodiment 37, wherein the vector is integrated into the genome of the immune cell.

39. The method of embodiment 38, wherein the vector is integrated into a TRAC locus of the genome.

40. A method of treating a disease or condition comprising administering the immune cell of any one of embodiments 1 to 24 to a subject.

41. A method of treating a subject having cancer, the method comprising:

administering a therapeutically effective amount of the immune cell of any of embodiments 1-24 to a subject in need thereof, whereby the immune cell induces killing of cancer cells in the subject.

42. A method of reducing T cell exhaustion comprising contacting a T cell with an autophagy modulator and/or a vector comprising a nucleotide sequence encoding the autophagy modulator.

43. A method of reducing NK cell exhaustion comprising contacting an NK cell with an autophagy modulator and/or a vector comprising a nucleotide sequence encoding the autophagy modulator.

44. A method of reducing T cell differentiation comprising contacting a T cell with an autophagy modulator and/or a vector comprising a nucleotide sequence encoding the autophagy modulator.

45. A method of reducing NK cell differentiation comprising contacting an NK cell with an autophagy modulator and/or a vector comprising a nucleotide sequence encoding the autophagy modulator.

46. A method of increasing T cell survival comprising contacting a T cell with an autophagy modulator and/or a vector comprising a nucleotide sequence encoding the autophagy modulator.

47. A method of increasing NK cell survival comprising contacting an NK cell with an autophagy modulator and/or a vector comprising a nucleotide sequence encoding the autophagy modulator.

48. A method of increasing the proliferation of an immune cell comprising contacting the immune cell with an autophagy modulator and/or a vector comprising a nucleotide sequence encoding the autophagy modulator.

49. A method of improving regulation of effector/memory differentiation of an immune cell, the method comprising contacting the immune cell with an autophagy modulator and/or a vector comprising a nucleotide sequence encoding the autophagy modulator.

50. A method of improving the mitochondrial function of an immune cell, the method comprising contacting the immune cell with an autophagy modulator, a vector comprising a nucleotide sequence encoding the autophagy modulator, and/or increasing autophagic metabolism (e.g. via an exogenous modulator such as a small or large molecule that promotes autophagic catabolism and/or enables autophagy-related anabolic processes).

51. The method of any one of embodiments 42 to 50, wherein the autophagy modulator is ATG1, ATG2, ATG3, ATG4, ATG5, ATG6, ATG7, ATG8, ATG8, ATG10, ATG11, ATG12, ATG13, ATG14, ATG15, ATG16, ATG17, ATG18, ATG19, ATG20, ATG21, ATG22, ATG23, ATG24, ATG25, ATG26, ATG27, ATG28, ATG29, ATG30, ATG31, ATG101, LC3, RAB7, VPS15, VPS34, VPS35, LC3I, LC3II, UVRAG, Beclin1, Protor, CAMKKbeta, BCL2, BCL-XL, AKT, ULK1, ULK2, ULK3, ULK4, DapK1, FIP200, TSC1, TSC2, STRAD, AMPK, Redd1, LKB, M025, PTEN, mTOR, Deptor, Rictor, Protor, PRAS40, LST8, Rheb, RAG A, RAG B, RAG C, RAG D, Raptor, PDK1, PI3K, IRS1, Insulin/IGF1 receptor, ERK, Rab40b, p53, DRAM1, NDFIP, MEK, RAF, SIN1, MAP4K3, Plac8, Dominant negative (DN) Rab7, Rab7, Dominant active (DA) Rab7, SLC7A5, or SLC3A2.

52. The method of any one of embodiments 42 to 51, wherein the autophagy modulator is ATG5.

53. The method of embodiment 52, wherein the ATG5 comprises an amino acid sequence at least 95% identical to the sequence of MTDDKDVLRDVWFGRIPTCFTLYQDEITEREAEPYYLLLPRVSYLTLVTDKVKKH FQKVMRQEDISEIWFEYEGTPLKWHYPIGLLFDLLASSSALPWNITVHFKSFPEKDL LHCPSKDAIEAHFMSCMKEADALKHKSQVINEMQKKDHKQLWMGLQNDRFDQF WAINRKLMEYPAEENGFRYIPFRIYQTTTERPFIQKLFRPVAADGQLHTLGDLLKE VCPSAIDPEDGEKKNQVMIHGIEPMLETPLQWLSEHLSYPDNFLHISIIPQPTD (SEQ ID NO: 1).

54. The method of embodiment 52, wherein the ATG5 comprises an amino acid sequence at least 95% identical to the sequence of MTDDKDVLRDVWFGRIPTCFTLYQDEITEREAEPYYLLLPRVSYLTLVTDKVKKH FQKVMRQEDVSEIWFEYEGTPLKWHYPIGLLFDLLASSSALPWNITVHFKSFPEKD LLHCPSKDAVEAHFMSCMKEADALKHKSQVINEMQKKDHKQLWMGLQNDRFD QFWAINRKLMEYPPEENGFRYIPFRIYQTTTERPFIQKLFRPVAADGQLHTLGDLLR EVCPSAVAPEDGEKRSQVMIHGIEPMLETPLQWLSEHLSYPDNFLHISIVPQPTD (SEQ ID NO: 2).

55. The method of any one of embodiments 42 to 51, wherein the autophagy modulator is ATG7.

56. The method of embodiment 55, wherein the ATG7 comprises an amino acid sequence at least 95% identical to the sequence of MAAATGDPGLSKLQFAPFSSALDVGFWHELTQKKLNEYRLDEAPKDIKGYYYNG DSAGLPARLTLEFSAFDMSAPTPARCCPAIGTLYNTNTLESFKTADKKLLLEQAAN EIWESIKSGTALENPVLLNKFLLLTFADLKKYHFYYWFCYPALCLPESLPLIQGPVG LDQRFSLKQIEALECAYDNLCQTEGVTALPYFLIKYDENMVLVSLLKHYSDFFQG QRTKITIGVYDPCNLAQYPGWPLRNFLVLAAHRWSSSFQSVEVVCFRDRTMQGA RDVAHSIIFEVKLPEMAFSPDCPKAVGWEKNQKGGMGPRMVNLSECMDPKRLAE SSVDLNLKLMCWRLVPTLDLDKVVSVKCLLLGAGTLGCNVARTLMGWGVRHIT FVDNAKISYSNPVRQPLYEFEDCLGGGKPKALAAADRLQKIFPGVNARGFNMSIP MPGHPVNFSSVTLEQARRDVEQLEQLIESHDVVFLLMDTRESRWLPAVIAASKRK LVINAALGFDTFVVMRHGLKKPKQQGAGDLCPNHPVASADLLGSSLFANIPGYKL GCYFCNDVVAPGDSTRDRTLDQQCTVSRPGLAVIAGALAVELMVSVLQHPEGGY AIASSSDDRMNEPPTSLGLVPHQIRGFLSRFDNVLPVSLAFDKCTACSSKVLDQYE REGFNFLAKVFNSSHSFLEDLTGLTLLHQETQAAEIWDMSDDETI (SEQ ID NO: 3).

57. The method of embodiment 55, wherein the ATG7 comprises an amino acid sequence at least 95% identical to the sequence of MGDPGLAKLQFAPFNSALDVGFWHELTQKKLNEYRLDEAPKDIKGYYYNGDSAG LPTRLTLEFSAFDMSASTPAHCCPAMGTLHNTNTLEAFKTADKKLLLEQSANEIW EAIKSGAALENPMLLNKFLLLTFADLKKYHFYYWFCCPALCLPESIPLIRGPVSLDQ RLSPKQIQALEHAYDDLCRAEGVTALPYFLFKYDDDTVLVSLLKHYSDFFQGQRT KITVGVYDPCNLAQYPGWPLRNFLVLAAHRWSGSFQSVEVLCFRDRTMQGARDV THSIIFEVKLPEMAFSPDCPKAVGWEKNQKGGMGPRMVNLSGCMDPKRLAESSV DLNLKLMCWRLVPTLDLDKVVSVKCLLLGAGTLGCNVARTLMGWGVRHVTFV DNAKISYSNPVRQPLYEFEDCLGGGKPKALAAAERLQKIFPGVNARGFNMSIPMP GHPVNFSDVTMEQARRDVEQLEQLIDNHDVIFLLMDTRESRWLPTVIAASKRKLV INAALGFDTFVVMRHGLKKPKQQGAGDLCPSHLVAPADLGSSLFANIPGYKLGCY FCNDVVAPGDSTRDRTLDQQCTVSRPGLAVIAGALAVELMVSVLQHPEGGYAIAS SSDDRMNEPPTSLGLVPHQIRGFLSRFDNVLPVSLAFDKCTACSPKVLDQYEREGF TFLAKVFNSSHSFLEDLTGLTLLHQETQAAEIWDMSDEETV (SEQ ID NO: 4).

58. The method of any one of embodiments 42 to 57, wherein the vector is a lentiviral vector.

### EXAMPLES

The following examples are provided to further describe some of the embodiments disclosed herein. The examples are intended to illustrate, not to limit, the disclosed embodiments.

### Example 1: Construction and Expression of ATG5 and ATG7 in BCMA-CAR T Cells

Materials and Methods

Lentiviruses were prepared from 15 cm 293-T cells as follows. Briefly, 11 million 293T cells were seeded onto collagen coated 15 cm dishes at day −1. At day 0, the following were transfected using EndoFectin Lenti (Genecopoeia): (i) 5.75 μg of lentiviral expression plasmid (a vector comprising sequencing encoding BCMA-CAR, ATG5-IRES-mcherry or ATG7-IRES-GFP), and (ii) 11.5 μl (0.5 μg/μl) of Lenti-Pac HIV mix (Genecopoeia) into 450 μl of Opti-MEM® I (Invitrogen). Sixteen hours later, the media was changed. After changing the media, viral supernatants were harvested at day 2 and day 3. The viruses were concentrated with Lenti-X concentrator (3:1 volume ratio, Clonetech, Cat #:63-123-1).

Lentiviral particles were titered by limiting dilution on SupT1 cells as follows. The virus was diluted from 1:3 to 1:1:6561. 50 μl of diluted lentivirus was added to SUPT1 cells (2e4) cultured in 96-well plate (100 μl/well). The cells were cultured for 3 days. The samples were then harvested and strained for FACS analysis. A graph of sample dilution versus sample titer was generated for each vector, and analyzed as follows. The curve should approach a slope of 0 (i.e. horizontal line) as the dilution increases and the percentage of positive cells falls below 20%. For each vector at each dilution, the titer was calculated according to the following formula: Titer (TU/ml)=(% positive/100)×2E4×20×dilution. (The 1st dilution at which the percentage of positive SupT1 cells is less than 20% was selected, and then the % positive cells calculated.)

BCMA-CAR T cells with ectopic expression of ATG5 or ATG7 were generated as follows. T cells were thawed and then resuspended at 1e6/ml in TexMACS media (Miltenyi). TransACT was added at a concentration of 57.14 μl TransACT/ml of cells at 1E6 cells/ml (1:17.5 dilution of TransACT). 2 ml/well was then plated into a 12-well plate, or 0.2 ml/well was plated into 96-well plate, followed by incubation at 37° C. One day later, varying volumes of one virus (ATG5; MOI=5) was added to one T cell population, varying volumes of the other virus (ATG7; MOI=5) was added to a second T cell population, the culture gently mixed, and the cells centrifuged at 2500 rpm for 2 hours at 30° C. A third T cell population was not transfected with either of ATG5 or ATG7. The cells were placed in an incubator for another day, and then varying volumes of another virus encoding BCMA-CAR (M01=5) was added to all three T cell populations. The culture was gently mixed and then centrifuged at 2500 rpm for 2 hrs at 30° C. The cells were cultured for another 8 days.

Lentiviral transduction efficiency was measured as follows. For analyzing BCMA CAR lentivirus transduction efficiency, cells were washed twice with staining buffer. The cell pellets were then resuspended with 100 μL of staining buffer. 2 μL of Fc block (BD Bioscience), BCMA-Fc-Biotin (1.25 ul/each, AcroBiosystems) and Near-IR Live/dead mix (Invitrogen) were added to wells, followed by incubation for 30 min at 4° C. in the dark. The cells were washed twice and then incubated with Streptavidin-APC (1:500) for 30 min at 4° C. in the dark. The cells were acquired on a FACS Fortessa (BD). Data processing for presentation was done using Flow Jo (Treestar Inc.) program. The population of APC⁺, GFP⁺ and mCherry⁺ represent the cells transduced with BCMA CAR, ATG7 and ATG5, respectively.

To examine the efficiency of ATG5 and ATG7 overexpression at the DNA level in T cells, quantitative reverse transcription polymerase chain reaction (qRT-PCR) was performed. An RNeasy Micro Kit (Qiagen) was used to extract RNA. The mRNA was then reverse transcribed to single strand complementary DNA (cDNA) with Superscript III First-Strand Synthesis System for RT PCR (Invitrogen). Real-time PCR was performed with an Applied Biosystem thermal Cycler. A SYBR-based protocol was used to detect gene expression (Applied Biosystems SYBR Green PCR Master Mix). The PCR reactions were performed in 96-well plates and run using the manufacture's recommended cycling parameters with triplicate (95° C. for 3 minutes, followed by 40 cycles of 95° C. for 15 seconds and 60° C. for 30 seconds). The cycle threshold (Ct) values for the genes of interest were normalized to the Ct for 18 s. 18 s serves as an internal control to quantify relative gene expression among samples tested. The Primers used for qRT-PCR were as follows: 18 s (forward primer: GGCCCTGTAATTGGAATGAGTC, reverse primer: CAAGATCCAACTACGAGCTT); the ATG5 and ATG7 primers were acquired from Genecopoeia.

A cytokine production assay was performed as follows. Effector cells (CAR T cells) were co-cultured with different target cells at Effector:Target cell ratio of 1:5 and 1:1. The cells were harvested after 16 hours. Golgi stop was added at the last 5 hours of culture. The cells were harvested and stained for the expression of IFN-γ and TNF-α.

A T cell proliferation assay was performed as follows. CAR T cell proliferation in response to BCMA-expressing target cells was evaluated. The target cell lines were BCMA positive multiple myeloma cell lines and NCI-H929-luc. CAR T cells were counted on a Cellometer. The target cells were stained with cell trace violet (10 μM, Invitrogen), irradiated at 50,000 rad and then co-cultured with CAR-T cells every seven days at the ratio of 1:1 and 5:1. As a negative control, medium alone was added to CAR-T cells. On day 7, cultured cells were stained for 20 mins with CD8-percp/cy5.5 and Near-IR Live/Dead and measured by flow cytometry. CAR expression was measured by two step incubation of each of (i) Biotinylated-BCMA-Fc and (ii) Streptavidin-APC for 20 mins each on ice. Flow cytometry data was acquired using BD Fortessa and analyzed by FlowJo software.

Results

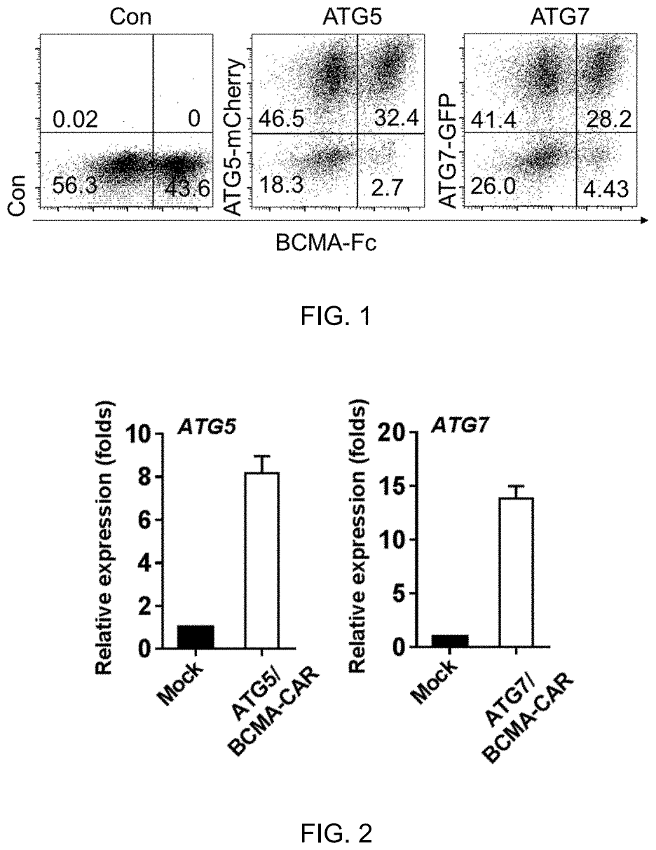

The overexpression of ATG5 and ATG7 in BCMA CAR-T cells was validated as follows. BMCA CAR-T cells expressing each of ATG5 and ATG7 were prepared according to above-described methods. The T-cells expressing ATG5 also expressed the mCherry marker, while the T-cells expressing ATG7 also expressed GFP. BMCA CAR-T cells expressing a scrambled vector were also prepared as a control. Lentiviral transduction efficiency was measured as described above. The data is shown in FIG. 1.

The data show that ATG5 ORF infected BCMA CAR-T cells express mCherry, and that ATG7 ORF infected BCMA CAR-T cells express GFP. GFP and mCherry expression were determined by FACS on day 6 after lentiviral infection. According to the observed mCherry and GFP expression, greater than 80% of T cells were transduced with ATG5 or ATG7.

To determine mRNA expression level of ATG5 and ATG7 in ATG5- and ATG7-lentivirus infected T cells, qRT-PCR experiment was performed. The data is shown in FIG. 2. As compared to the scramble vector (“Mock”), there was an approximately 8-fold increase in ATG5 gene expression and an approximately 14-fold increase of ATG7 gene expression. This data indicates that lentivirus was capable of increasing mRNA levels of ATG5 and ATG7.

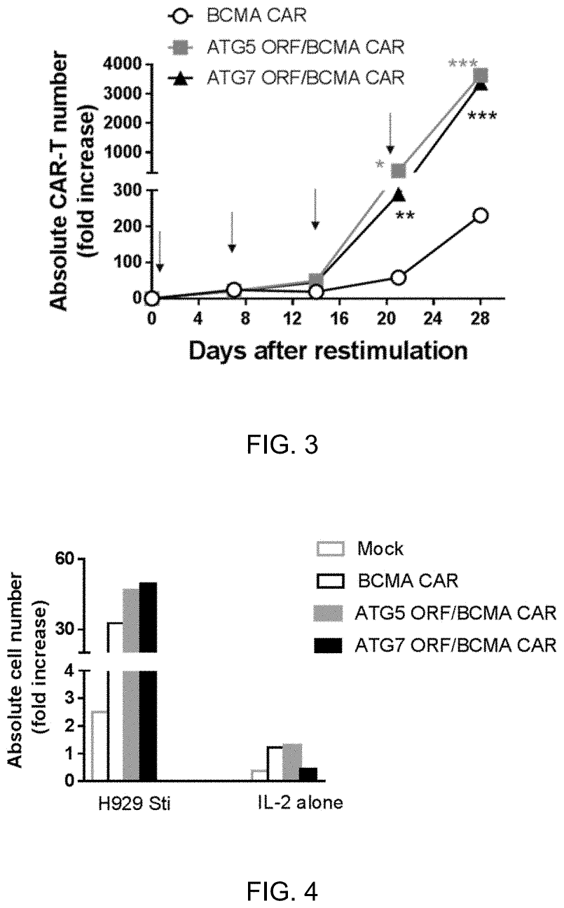

Ectopic expression of ATG5 or ATG7 increases CAR-T cell expansion upon repeated stimulation. The proliferation of donor BCMA-CAR cells was measured in response to repetitive stimulation with BCMA-expressing H929 cells. Pan-T cells were transduced with either the BCMA-CAR alone, BCMA-CAR+ATG5, or BCMA-CAR+ATG7. Cells were stimulated with H929 cells (effector:target ratio 1:5) every seven days. The total cells per mL were counted by Beckman Coulter Vi-Cell. The CAR-T cell number was calculated by total cell count multiplying CAR percentage analyzed by flow cytometry. The data is shown in FIG. 3, with the arrows indicating each day where stimulation occurred. This data indicates that ectopic expression of ATG5 or ATG7 could enhance BCMA CAR-T expansion upon repeated tumor antigenic stimulation. Arrow indicates each stimulation. Shown are representative experiments of technical triplicates. P values were determined using a two-tailed Student's t-test. *P<0.05, **P<0.01, and ***P<0.001.

Ectopic expression of ATG5 or ATG7 does not increase BCMA CAR-T expansion in the absence of antigenic stimulation. BCMA CAR-T cells (transduced with ATG5 or ATG7) were cultured in the presence of IL-2, or upon stimulation by BCMA-expressing H929 cells. CAR-T cell number was counted, with the data shown in FIG. 4. Untransduced T cells (Mock) were included as a control for non-specific cell expansion. As shown in FIG. 4, overexpression of ATG5 or ATG7 augments tumor Ag-driven T proliferation, but not cytokine-induced proliferation.

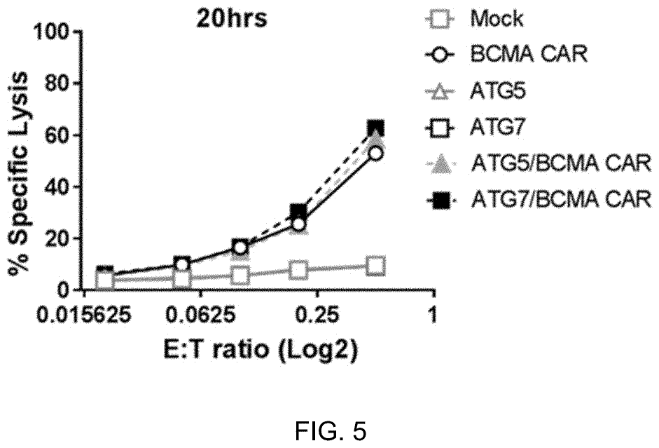

Ectopic expression of ATG5 and ATG7 does not impair CAR T cell cytotoxic activity. The cytotoxic capacity of BCMA-CAR cells (transduced with ATG5 or ATG7) was measured after overnight co-culture with luciferase expressing H929 cells (one representative experiment with technical duplicates). Untransduced T cells (Mock), and ATG5- and ATG7-transduced T cells were included as an additional group to control for non-specific lysis. The data is shown in FIG. 5, and indicate that overexpression of ATG5 or ATG7 does not impact CAR T cytotoxic capability.

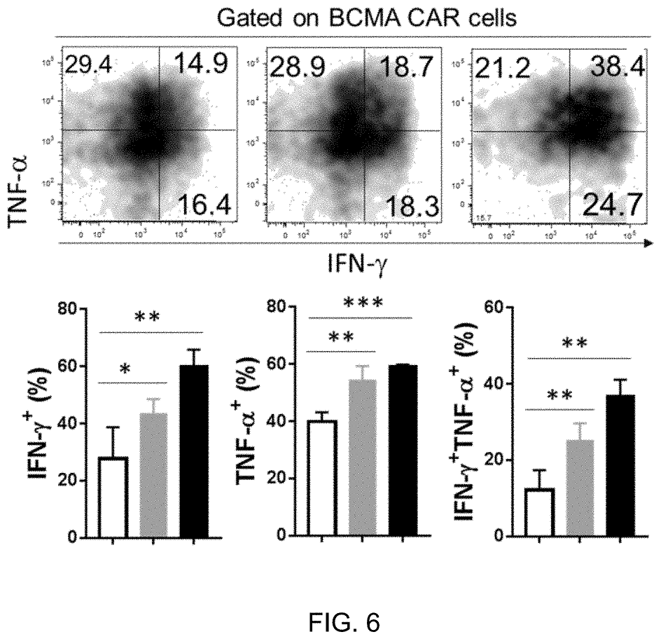

The effect of ectopic expression of ATG5 or ATG7 on IFN-γ- and TNF-α-producing CAR-T cells was assayed as follows. Multiple myeloma patient derived BCMA-CAR cells were co-cultured with BCMA-positive cells (H929, E:T=1:5) for 16 hours. The cytokine production of the BCMA-CAR cells was measured (with data from one representative experiment of 3-4 technical replicates shown). The data is shown in FIG. 6, with the representative flow cytometry plots showing the expression of IFN-γ and TNF-α. Bar graphs showed the fraction of IFN-γ⁺, TNF-α⁺ and IFN-γ⁺ TNF-α⁺ cells. fine frequencies of gated populations. The data indicate that overexpression of ATG5 and ATG7 could significantly increase CAR T cell production of effector cytokine IFN-γ and TNF-α. *P<0.05, **P<0.01, and ***P<0.001.

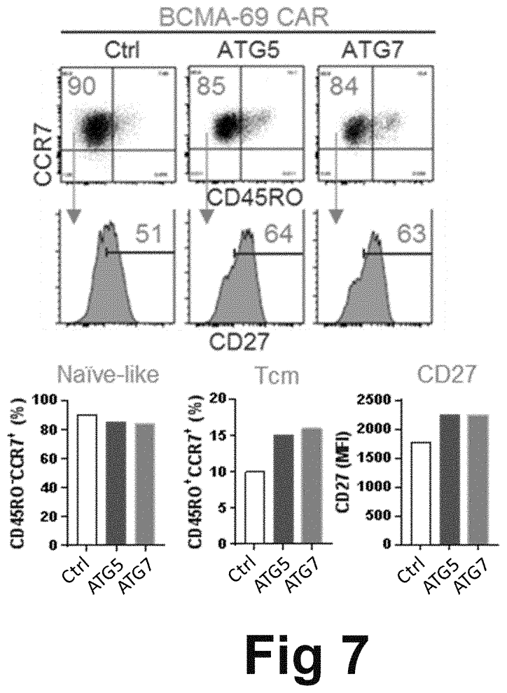

Ectopic expression of ATG5 and ATG7 in BCMA-CAR cells did not significantly affect CAR-T cell differentiation during initial activation. This data is shown in FIG. 7, with flow cytometry plots showing the expression of CD45RO, CCR7, and CD27 in each of BCMA-CAR cells, BCMA-CAR cells ectopically expressing ATG5, and BCMA-CAR cells ectopically expressing ATG7. The Pan-T cells were stimulated with CD3/CD28 Ab in the presence of IL-2, followed by lentiviral transduction of BCMA-CAR and ATG5 or ATG7, and then analyzed at day 10 after stimulation. The data indicate that overexpression of ATG5 and ATG7 does not significantly affect CAR-T differentiation during initial activation.

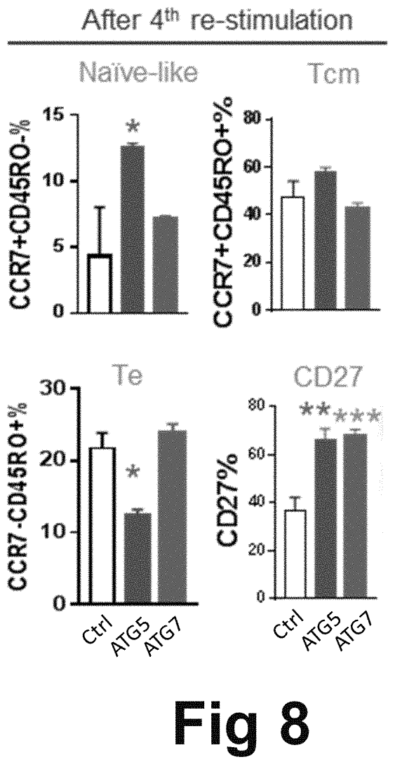

Also, ectopic expression of ATG5 and ATG7 in BCMA-CAR cells was shown to promote the generation of long-lasting less differentiated CAR-T cells. This data is shown in FIG. 8, with representative graphs showing the expression of ATG5 and ATG7 in fractions of CD45RO⁻CCR7⁺ (naïve like), CD45RO⁺CCR7⁺ (Tcm), CD45RO⁺CCR7⁻ (Te) and CD27-expressing cells. The cells were stimulated with BCMA-expressing H929 cells at days 0, 7, 14, and 21. This repeated stimulation induced CAR-T cell differentiation into effector T cells and reduced their expression of CD27. However, CAR-T cells expressing ATG5 had a naïve-like phenotype. This phenotype differed from T cells expressing BCMA-CAR alone, whose phenotypes were dominated by effector/effector memory cells. In addition, the expression of CD27 was significantly increased in cells expressing BCMA-CAR with either of ATG5 and ATG7. This data indicated that ectopic expression of ATG5 or ATG7 could promote the generation of long-lasting, less differentiated CAR-T cells. *P<0.05, **P<0.01, and ***P<0.001.

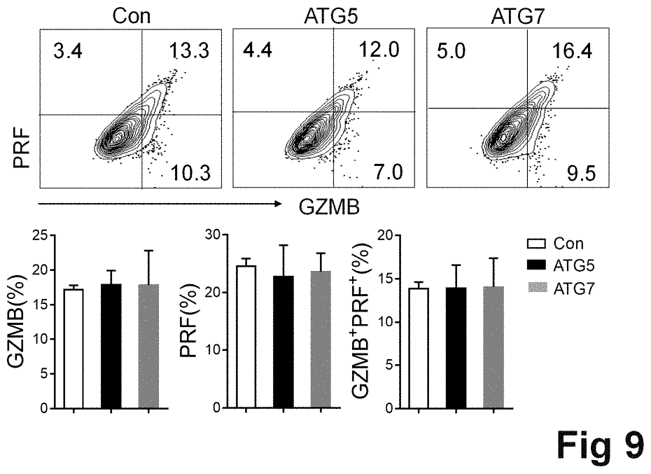

Ectopic expression of ATG5 and ATG7 does not impair CAR T cell cytotoxic activity as measured by the expression of Granzyme B (GZMB) and Perforin (PRF). FIG. 9 shows the expression of each of these cytotoxic molecules in each of BCMA-CAR cells, BCMA-CAR cells ectopically expressing ATG5, and BCMA-CAR cells ectopically expressing ATG7. The data indicate that overexpression of ATG5 and ATG7 did not impair CAR-T cell production of GZMB or PRF, and also that overexpression of ATG5 or ATG7 does not impact CAR T cytotoxic capability (see also FIG. 5).

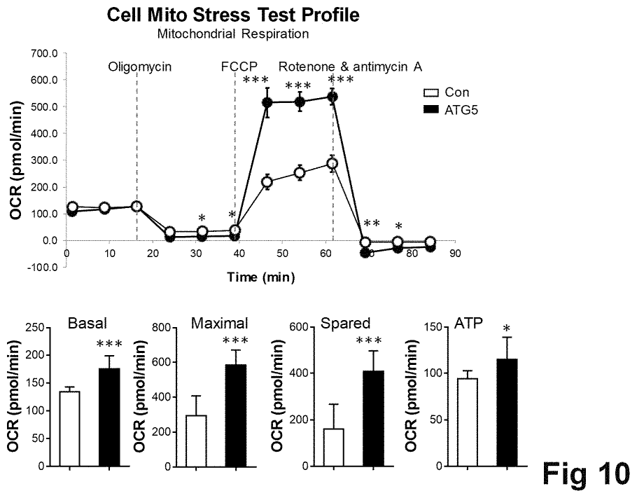

ATG5 is capable of improving cells' mitochondrial function. FIG. 10 demonstrates the representative oxygen consumption rates (OCR) of Jurkat cells (transduced with or without ATG5). The OCR was measured prior to the addition of drugs (basal OCR) and then following the addition of the indicated drugs. Based on the increase in OCR at all stages of the mitochondrial stress test in cells overexpressing ATG5, overexpression of ATG5 significantly augmented cell mitochondrial function. *P<0.05, **P<0.01, and ***P<0.001.

The teachings of all patents, published applications and references cited herein are incorporated by reference in their entirety.

While example embodiments have been particularly shown and described, it will be understood by those skilled in the art that various changes in form and details may be made therein without departing from the scope of the embodiments encompassed by the appended claims.

SEQUENCE LISTING

SEQ ID NO: 1 - human ATG5 sequence
MTDDKDVLRDVWFGRIPTCFTLYQDEITEREAEPYYLLLPRVSYLTLVTDKVKKH
FQKVMRQEDISEIWFEYEGTPLKWHYPIGLLFDLLASSSALPWNITVHFKSFPEKDL
LHCPSKDAIEAHFMSCMKEADALKHKSQVINEMQKKDHKQLWMGLQNDRFDQF
WAINRKLMEYPAEENGFRYIPFRIYQTTTERPFIQKLFRPVAADGQLHTLGDLLKE
VCPSAIDPEDGEKKNQVMIHGIEPMLETPLQWLSEHLSYPDNFLHISIIPQPTD
SEQ ID NO: 2 - mouse ATG5 sequence
MTDDKDVLRDVWFGRIPTCFTLYQDEITEREAEPYYLLLPRVSYLTLVTDKVKKH
FQKVMRQEDVSEIWFEYEGTPLKWHYPIGLLFDLLASSSALPWNITVHFKSFPEKD
LLHCPSKDAVEAHFMSCMKEADALKHKSQVINEMQKKDHKQLWMGLQNDRFD
QFWAINRKLMEYPPEENGFRYIPFRIYQTTTERPFIQKLFRPVAADGQLHTLGDLLR
EVCPSAVAPEDGEKRSQVMIHGIEPMLETPLQWLSEHLSYPDNFLHISIVPQPTD
SEQ ID NO: 3 - human ATG7 sequence
MAAATGDPGLSKLQFAPFSSALDVGFWHELTQKKLNEYRLDEAPKDIKGYYYNG
DSAGLPARLTLEFSAFDMSAPTPARCCPAIGTLYNTNTLESFKTADKKLLLEQAAN
EIWESIKSGTALENPVLLNKFLLLTFADLKKYHFYYWFCYPALCLPESLPLIQGPVG
LDQRFSLKQIEALECAYDNLCQTEGVTALPYFLIKYDENMVLVSLLKHYSDFFQG
QRTKITIGVYDPCNLAQYPGWPLRNFLVLAAHRWSSSFQSVEVVCFRDRTMQGA
RDVAHSIIFEVKLPEMAFSPDCPKAVGWEKNQKGGMGPRMVNLSECMDPKRLAE
SSVDLNLKLMCWRLVPTLDLDKVVSVKCLLLGAGTLGCNVARTLMGWGVRHIT
FVDNAKISYSNPVRQPLYEFEDCLGGGKPKALAAADRLQKIFPGVNARGFNMSIP
MPGHPVNFSSVTLEQARRDVEQLEQLIESHDVVFLLMDTRESRWLPAVIAASKRK
LVINAALGFDTFVVMRHGLKKPKQQGAGDLCPNHPVASADLLGSSLFANIPGYKL
GCYFCNDVVAPGDSTRDRTLDQQCTVSRPGLAVIAGALAVELMVSVLQHPEGGY
AIASSSDDRMNEPPTSLGLVPHQIRGFLSRFDNVLPVSLAFDKCTACSSKVLDQYE
REGFNFLAKVFNSSHSFLEDLTGLTLLHQETQAAEIWDMSDDETI
SEQ ID NO: 4 - mouse ATG7 sequence
MGDPGLAKLQFAPFNSALDVGFWHELTQKKLNEYRLDEAPKDIKGYYYNGDSAG
LPTRLTLEFSAFDMSASTPAHCCPAMGTLHNTNTLEAFKTADKKLLLEQSANEIW
EAIKSGAALENPMLLNKFLLLTFADLKKYHFYYWFCCPALCLPESIPLIRGPVSLDQ
RLSPKQIQALEHAYDDLCRAEGVTALPYFLFKYDDDTVLVSLLKHYSDFFQGQRT
KITVGVYDPCNLAQYPGWPLRNFLVLAAHRWSGSFQSVEVLCFRDRTMQGARDV
THSIIFEVKLPEMAFSPDCPKAVGWEKNQKGGMGPRMVNLSGCMDPKRLAESSV
DLNLKLMCWRLVPTLDLDKVVSVKCLLLGAGTLGCNVARTLMGWGVRHVTFV
DNAKISYSNPVRQPLYEFEDCLGGGKPKALAAAERLQKIFPGVNARGFNMSIPMP
GHPVNFSDVTMEQARRDVEQLEQLIDNHDVIFLLMDTRESRWLPTVIAASKRKLV
INAALGFDTFVVMRHGLKKPKQQGAGDLCPSHLVAPADLGSSLFANIPGYKLGCY
FCNDVVAPGDSTRDRTLDQQCTVSRPGLAVIAGALAVELMVSVLQHPEGGYAIAS
SSDDRMNEPPTSLGLVPHQIRGFLSRFDNVLPVSLAFDKCTACSPKVLDQYEREGF
TFLAKVFNSSHSFLEDLTGLTLLHQETQAAEIWDMSDEETV
SEQ ID NO: 5 - human ATG5 sequence (cDNA)
GTCTGGACTTGTGGTGCGCTGCCAGGGATCCGCAGCGTTGCCGGTTGTATTCG
CTGGATACCAGAGGGCGGAAGTGCAGCAGGGTTCAGCTCCGACCTCCGCGCC
GGTGCTTTTTGCGGCTGCGCGGGCTTCCTGGAGTCCTGCTACCGCGTCCCCGCA
GGACAGTGTGTCAGGCGGGCAGCTTGCCCCGCCGCCCCACCGGAGCGCGGAA
TCTGGGCGTCCCCACCAGTGCGGGGAGCCGGAAGGAGGAGCCATAGCTTGGA
GTAGGTTTGGCTTTGGTTGAAATAAGAATTTAGCCTGTATGTACTGCTTTAACT
CCTGGAAGAATGACAGATGACAAAGATGTGCTTCGAGATGTGTGGTTTGGACG
AATTCCAACTTGTTTCACGCTATATCAGGATGAGATAACTGAAAGGGAAGCAG
AACCATACTATTTGCTTTTGCCAAGAGTAAGTTATTTGACGTTGGTAACTGACA
AAGTGAAAAAGCACTTTCAGAAGGTTATGAGACAAGAAGACATTAGTGAGAT
ATGGTTTGAATATGAAGGCACACCACTGAAATGGCATTATCCAATTGGTTTGC
TATTTGATCTTCTTGCATCAAGTTCAGCTCTTCCTTGGAACATCACAGTACATT
TTAAGAGTTTTCCAGAAAAAGACCTTCTGCACTGTCCATCTAAGGATGCAATT
GAAGCTCATTTTATGTCATGTATGAAAGAAGCTGATGCTTTAAAACATAAAAG
TCAAGTAATCAATGAAATGCAGAAAAAAGATCACAAGCAACTCTGGATGGGA
TTGCAAAATGACAGATTTGACCAGTTTTGGGCCATCAATCGGAAACTCATGGA
ATATCCTGCAGAAGAAAATGGATTTCGTTATATCCCCTTTAGAATATATCAGA
CAACGACTGAAAGACCTTTCATTCAGAAGCTGTTTCGTCCTGTGGCTGCAGAT
GGACAGTTGCACACACTAGGAGATCTCCTCAAAGAAGTTTGTCCTTC
SEQ ID NO: 6 - mouse ATG5 sequence (cDNA)
ATGACAGATGACAAAGATGTGCTTCGAGATGTGTGGTTTGGACGAATTCCAAC
TTGCTTTACTCTCTATCAGGATGAGATAACTGAAAGAGAAGCAGAACCATACT
ATTTGCTTTTGCCAAGAGTCAGCTATTTGACGTTGGTAACTGACAAAGTGAAA
AAGCACTTTCAGAAGGTTATGAGACAAGAAGATGTTAGTGAGATATGGTTTGA
ATATGAAGGCACACCCCTGAAATGGCATTATCCAATTGGTTTACTATTTGATCT
TCTTGCATCAAGTTCAGCTCTTCCTTGGAACATCACAGTACATTTCAAGAGTTT
TCCAGAAAAGGACCTTCTACACTGTCCATCCAAGGATGCGGTTGAGGCTCACT
TTATGTCGTGTATGAAAGAAGCTGATGCTTTAAAGCATAAAAGTCAAGTGATC
AACGAAATGCAGAAAAAAGACCACAAGCAGCTCTGGATGGGACTGCAGAATG
ACAGATTTGACCAGTTTTGGGCCATCAACCGGAAACTCATGGAATATCCTCCA
GAAGAAAATGGATTTCGTTATATCCCCTTTAGAATATATCAGACCACGACGGA
GCGGCCTTTCATCCAGAAGCTGTTCCGGCCTGTGGCCGCAGATGGACAGCTGC
ACACACTTGGAGATCTCCTCAGAGAAGTCTGTCCTTCCGCAGTCGCCCCTGAA
GATGGAGAGAAGAGGAGCCAGGTGATGATTCACGGGATAGAGCCAATGCTGG
AAACCCCTCTGCAGTGGCTGAGCGAGCATCTGAGCTACCCAGATAACTTTCTT
CATATTAGCATTGTCCCCCAGCCAACAGATTGA
SEQ ID NO: 7 - human ATG7 sequence (cDNA)
GGAAGTTGAGCGGCGGCAAGAAATAATGGCGGCAGCTACGGGGGATCCTGGA
CTCTCTAAACTGCAGTTTGCCCCTTTTAGTAGTGCCTTGGATGTTGGGTTTTGG
CATGAGTTGACCCAGAAGAAGCTGAACGAGTATCGGCTGGATGAAGCTCCCA
AGGACATTAAGGGTTATTACTACAATGGTGACTCTGCTGGGCTGCCAGCTCGC
TTAACATTGGAGTTCAGTGCTTTTGACATGAGTGCTCCCACCCCAGCCCGTTGC
TGCCCAGCTATTGGAACACTGTATAACACCAACACACTCGAGTCTTTCAAGAC
TGCAGATAAGAAGCTCCTTTTGGAACAAGCAGCAAATGAGATATGGGAATCC
ATAAAATCAGGCACTGCTCTTGAAAACCCTGTACTCCTCAACAAGTTCCTCCTC
TTGACATTTGCAGATCTAAAGAAGTACCACTTCTACTATTGGTTTTGCTATCCT
GCCCTCTGTCTTCCAGAGAGTTTACCTCTCATTCAGGGGCCAGTGGGTTTGGAT
CAAAGGTTTTCACTAAAACAGATTGAAGCACTAGAGTGTGCATATGATAATCT
TTGTCAAACAGAAGGAGTCACAGCTCTTCCTTACTTCTTAATCAAGTATGATGA
GAACATGGTGCTGGTTTCCTTGCTTAAACACTACAGTGATTTCTTCCAAGGTCA
AAGGACGAAGATAACAATTGGTGTATATGATCCCTGTAACTTAGCCCAGTACC
CTGGATGGCCTTTGAGGAATTTTTTGGTCCTAGCAGCCCACAGATGGAGTAGC
AGTTTCCAGTCTGTTGAAGTTGTTTGCTTCCGTGACCGTACCATGCAGGGGGCG
AGAGACGTTGCCCACAGCATCATCTTCGAAGTGAAGCTTCCAGAAATGGCATT
TAGCCCAGATTGTCCTAAAGCAGTTGGATGGGAAAAGAACCAGAAAGGAGGC
ATGGGACCAAGGATGGTGAACCTCAGTGAATGTATGGACCCT
SEQ ID NO: 8 - mouse ATG7 sequence (cDNA)
ATGGGGGACCCTGGACTGGCCAAGTTGCAGTTCGCCCCCTTTAATAGTGCCCT
GGACGTTGGCCTCTGGCACGAACTGACCCAGAAGAAGTTGAACGAGTACCGC
CTGGACGAGGCACCCAAAGACATCAAGGGCTATTACTACAATGGTGACTCTGC
TGGTCTGCCCACCCGCTTGACGTTGGAGTTCAGTGCTTTTGACATGAGTGCCTC
CACGCCTGCCCACTGCTGCCCGGCCATGGGAACCCTGCACAACACCAACACAC
TTGAGGCTTTTAAGACAGCAGACAAGAAGCTCCTTCTGGAGCAGTCAGCAAAT
GAGATCTGGGAAGCCATAAAGTCAGGTGCTGCTCTCGAAAACCCCATGCTCCT
CAACAAGTTTCTGCTCCTGACCTTCGCGGACCTAAAGAAGTACCACTTCTACTA
CTGGTTTTGCTGCCCCGCCCTCTGTCTTCCTGAGAGCATCCCTCTAATCCGGGG
ACCTGTGAGCTTGGATCAAAGGCTTTCACCAAAACAGATCCAGGCCCTGGAGC
ATGCCTATGATGATCTGTGTCGAGCCGAAGGCGTCACGGCCCTGCCCTACTTCT
TATTCAAGTACGATGACGACACTGTTCTGGTCTCCTTGCTCAAACACTACAGTG
ATTTCTTCCAAGGTCAAAGGACAAAGATAACAGTTGGTGTGTACGATCCCTGT
AACCTAGCCCAGTACCCTGGATGGCCTTTGAGGAATTTTTTGGTCCTGGCAGCC
CACAGATGGAGCGGCAGTTTCCAGTCCGTTGAAGTCCTCTGCTTTCGGGACCG
CACCATGCAGGGAGCTAGAGACGTGACACATAGCATCATCTTTGAAGTGAAAC
TTCCAGAAATGGCATTTAGCCCAGATTGTCCTAAAGCTGTTGGCTGGGAGAAG
AACCAGAAAGGAGGCATGGGTCCGAGGATGGTGAACCTCAGTGGATGTATGG
ACCCCAAAAGGCTGGCTGAGTCATCTGTGGATCTGAATCTCA

What is claimed is:

1. An immune cell expressing a first vector comprising a nucleotide sequence encoding a chimeric antigen receptor (CAR) and a) a second vector comprising a nucleotide sequence encoding an autophagy modulator or b) a second nucleotide sequence encoding an autophagy modulator.

2. An immune cell comprising a vector comprising a nucleotide sequence encoding an autophagy modulator.

3. The immune cell of claim 2, wherein the immune cell further comprises a CAR.

4. The immune cell of claim 1, wherein the genome of the immune cell comprises one or more additional autophagy modulator genes.

5. The immune cell of claim 1, wherein:
a) a promoter of an autophagy modulator gene is replaced with a constitutive promoter;
b) an enhancer sequence of an autophagy modulator gene is replaced with a second enhancer sequence that is effective to increase transcription of the autophagy modulator gene; or
c) both a) and b).

6. The immune cell of claim 1, wherein the immune cell is a lymphocyte.

7. The immune cell of claim 6, wherein the immune cell is a tumor penetrating lymphocyte.

8. The immune cell of claim 6, wherein the immune cell is a T cell or a Natural Killer (NK) cell.

9. The immune cell of claim 1, wherein the CAR comprises an extracellular domain that specifically binds to the B-cell maturation antigen (BCMA), a CD19 antigen, a CD30 antigen, a CD123 antigen, an FLT3 antigen, and kallikrein-2 antigen.

10. The immune cell of claim 1, wherein the autophagy modulator is ATG1, ATG2, ATG3, ATG4, ATG5, ATG6, ATG7, ATG8, ATG8, ATG10, ATG11, ATG12, ATG13, ATG14, ATG15, ATG16, ATG17, ATG18, ATG19, ATG20, ATG21, ATG22, ATG23, ATG24, ATG25, ATG26, ATG27, ATG28, ATG29, ATG30, ATG31, ATG101, LC3, RAB7, VPS15, VPS34, VPS35, LC3I, LC3II, UVRAG, Beclin1, Protor, CAMKKbeta, BCL2, BCL-XL, AKT, ULK1, ULK2, ULK3, ULK4, DapK1, FIP200, TSC1, TSC2, STRAD, AMPK, Redd1, LKB, M025, PTEN, mTOR, Deptor, Rictor, Protor, PRAS40, LST8, Rheb, RAG A, RAG B, RAG C, RAG D, Raptor, PDK1, PI3K, IRS1, Insulin/IGF1 receptor, ERK, Rab40b, p53, DRAM1, NDFIP, MEK, RAF, SIN1, MAP4K3, Plac8, Dominant negative (DN) Rab7, Rab7, Dominant active (DA) Rab7, SLC7A5, or SLC3A2.

11. The immune cell of claim 10, wherein the autophagy modulator is ATG5.

12. The immune cell of claim 11, wherein:
a) the ATG5 comprises an amino acid sequence at least 95% identical to the sequence of MTDDKDVLRDVWFGRIPTCFTLYQDEITEREAEPYYLLLPRVSYLTLVTDKVKKH FQKVMRQEDISEIWFEYEGTPLKWHYPIGLLFDLLASSSALPWNITVHFKSFPEKDL LHCPSKDAIEAHFMSCMKEADALKHKSQVINEMQKKDHKQLWMGLQNDRFDQF WAINRKLMEYPAEENGFRYIPFRIYQTTTERPFIQKLFRPVAADGQLHTLGDLLKE VCPSAIDPEDGEKKNQVMIHGIEPMLETPLQWLSEHLSYPDNFLHISIIPQPTD (SEQ ID NO: 1) or
b) the ATG5 comprises an amino acid sequence at least 95% identical to the sequence of MTDDKDVLRDVWFGRIPTCFTLYQDEITEREAEPYYLLLPRVSYLTLVTDKVKKH FQKVMRQEDVSEIWFEYEGTPLKWHYPIGLLFDLLASSSALPWNITVHFKSFPEKD LLHCPSKDAVEAHFMSCMKEADALKHKSQVINEMQKKDHKQLWMGLQNDRFD QFWAINRKLMEYPPEENGFRYIPFRIYQTTTERPFIQKLFRPVAADGQLHTLGDLLR EVCPSAVAPEDGEKRSQVMIHGIEPMLETPLQWLSEHLSYPDNFLHISIVPQPTD (SEQ ID NO: 2).

13. The immune cell of claim 10, wherein the autophagy modulator is ATG7.

14. The immune cell of claim 13, wherein a) the ATG7 comprises an amino acid sequence at least 95% identical to the sequence of MAAATGDPGLSKLQFAPFSSALDVGFWHELTQKKLNEYRLDEAPKDIKGYYYNG DSAGLPARLTLEFSAFDMSAPTPARCCPAIGTLYNTNTLESFKTADKKLLLEQAAN EIWESIKSGTALENPVLLNKFLLLTFADLKKYHFYYWFCYPALCLPESLPLIQGPVG LDQRFSLKQIEALECAYDNLCQTEGVTALPYFLIKYDENMVLVSLLKHYSDFFQG QRTKITIGVYDPCNLAQYPGWPLRNFLVLAAHRWSSSFQSVEVVCFRDRTMQGA RDVAHSIIFEVKLPEMAFSPDCPKAVGWEKNQKGGMGPRMVNLSECMDPKRLAE SSVDLNLKLMCWRLVPTLDLDKVVSVKCLLLGAGTLGCNVARTLMGWGVRHIT FVDNAKISYSNPVRQPLYEFEDCLGGGKPKALAAADRLQKIFPGVNARGFNMSIP MPGHPVNFSSVTLEQARRDVEQLEQLIESHDVVFLLMDTRESRWLPAVIAASKRK LVINAALGFDTFVVMRHGLKKPKQQGAGDLCPNHPVASADLLGSSLFANIPGYKL GCYFCNDVVAPGDSTRDRTLDQQCTVSRPGLAVIAGALAVELMVSVLQHPEGGY AIASSSDDRMNEPPTSLGLVPHQIRGFLSRFDNVLPVSLAFDKCTACSSKVLDQYE REGFNFLAKVFNSSHSFLEDLTGLTLLHQETQAAEIWDMSDDETI (SEQ ID NO: 3) or
b) the ATG7 comprises an amino acid sequence at least 95% identical to the sequence of MGDPGLAKLQFAPFNSALDVGFWHELTQKKLNEYRLDEAPKDIKGYYYNGDSAG LPTRLTLEFSAFDMSASTPAHCCPAMGTLHNTNTLEAFKTADKKLLLEQSANEIW EAIKSGAALENPMLLNKFLLLTFADLKKYHFYYWFCCPALCLPESIPLIRGPVSLDQ RLSPKQIQALEHAYDDLCRAEGVTALPYFLFKYDDDTVLVSLLKHYSDFFQGQRT KITVGVYDPCNLAQYPGWPLRNFLVLAAHRWSGSFQSVEVLCFRDRTMQGARDV THSIIFEVKLPEMAFSPDCPKAVGWEKNQKGGMGPRMVNLSGCMDPKRLAESSV DLNLKLMCWRLVPTLDLDKVVSVKCLLLGAGTLGCNVARTLMGWGVRHVTFV DNAKISYSNPVRQPLYEFEDCLGGGKPKALAAAERLQKIFPGVNARGFNMSIPMP GHPVNFSDVTMEQARRDVEQLEQLIDNHDVIFLLMDTRESRWLPTVIAASKRKLV INAALGFDTFVVMRHGLKKPKQQGAGDLCPSHLVAPADLGSSLFANIPGYKLGCY FCNDVVAPGDSTRDRTLDQQCTVSRPGLAVIAGALAVELMVSVLQHPEGGYAIAS SSDDRMNEPPTSLGLVPHQIRGFLSRFDNVLPVSLAFDKCTACSPKVLDQYEREGF TFLAKVFNSSHSFLEDLTGLTLLHQETQAAEIWDMSDEETV (SEQ ID NO: 4).

15. The immune cell of claim 1, wherein the vector is a lentiviral vector.

16. The immune cell of claim 1, wherein:
a) the expression of the autophagy modulator is at least four times the level of expression of the autophagy modulator in a comparable immune cell with normal expression of the autophagy inhibitor;
b) the cytotoxic activity of the immune cell is not lower than that of a comparable immune cell with normal expression of the autophagy inhibitor;
c) the immune cell is able to proliferate to a greater extent than a comparable immune cell with normal expression of the autophagy inhibitor; or
d) any combination of a)-c).

17. The immune cell of claim 1, wherein:
a) the immune cell enters a state of T cell exhaustion at a later time than a comparable immune cell with normal expression of the autophagy inhibitor; or
b) the immune cell does not undergo T cell exhaustion.

18. A pharmaceutical composition comprising an effective amount of the immune cell of claim 1 and a pharmaceutically acceptable excipient.

19. A method of preparing the immune cells of claim 1, the method comprising introducing the first and second vectors into an immune cell.

20. The method of claim 19, wherein:
a) the first vector, the second vector, or both the first and second vectors are transduced into the immune cell; or
b) a gene editing system is used to introduce the first vector and/or the second vector into the immune cell.

21. The method of claim 20, wherein the first vector is a viral vector, the second vector is a viral vector, or both the first vector and the second vector are viral vectors.

22. The method of claim 20, wherein the gene editing system is selected from the group consisting of a CRISPR/Cas9 system, a CRISPR/Cpf1 system a zinc finger nuclease system, a TALEN system, and a meganuclease system.

23. The method of claim 20, wherein the first vector and/or the second vector is integrated into the genome of the immune cell.

24. The method of claim 23, wherein the first vector and/or the second vector is integrated into a TRAC locus of the genome.

25. A method of preparing the immune cells of claim 1, the method comprising introducing the vector into an immune cell.

26. The method of claim 25, wherein:
a) the vector is transduced into the immune cell or
b) a gene editing system is used to introduce the vector into the immune cell.

27. The method of claim 25, wherein the vector is a viral vector.

28. The method of claim 26, wherein the gene editing system is selected from the group consisting of a CRISPR/Cas9 system, a CRISPR/Cpf1 system a zinc finger nuclease system, a TALEN system, and a meganuclease system.

29. The method of claim 26, wherein the vector is integrated into the genome of the immune cell.

30. The method of claim 29, wherein the vector is integrated into a TRAC locus of the genome.

31. A method of treating a disease or condition comprising administering the immune cell of claim 1 to a subject.

32. A method of treating a subject having cancer, the method comprising administering a therapeutically effective amount of the immune cell of claim 1 to a subject in need thereof, whereby the immune cell induces killing of cancer cells in the subject.

33. A method of reducing T cell or NK cell exhaustion comprising contacting a T cell or NK cell with an autophagy modulator and/or a vector comprising a nucleotide sequence encoding the autophagy modulator.

34. A method of increasing the proliferation of an immune cell, improving regulation of effector/memory differentiation of an immune cell, and/or improving the mitochondrial function of an immune cell comprising contacting the immune cell with an autophagy modulator and/or a vector comprising a nucleotide sequence encoding the autophagy modulator.

35. The method of claim 33, wherein the autophagy modulator is ATG1, ATG2, ATG3, ATG4, ATG5, ATG6, ATG7, ATG8, ATG8, ATG10, ATG11, ATG12, ATG13, ATG14, ATG15, ATG16, ATG17, ATG18, ATG19, ATG20, ATG21, ATG22, ATG23, ATG24, ATG25, ATG26, ATG27, ATG28, ATG29, ATG30, ATG31, ATG101, LC3, RAB7, VPS15, VPS34, VPS35, LC3I, LC3II, UVRAG, Beclin1, Protor, CAMKKbeta, BCL2, BCL-XL, AKT, ULK1, ULK2, ULK3, ULK4, DapK1, FIP200, TSC1, TSC2, STRAD, AMPK, Redd1, LKB, M025, PTEN, mTOR, Deptor, Rictor, Protor, PRAS40, LST8, Rheb, RAG A, RAG B, RAG C, RAG D, Raptor, PDK1, PI3K, IRS1, Insulin/IGF1 receptor, ERK, Rab40b, p53, DRAM1, NDFIP, MEK, RAF, SIN1, MAP4K3, Plac8, Dominant negative (DN) Rab7, Rab7, Dominant active (DA) Rab7, SLC7A5, or SLC3A2.

36. The method of claim 35, wherein the autophagy modulator is ATG5.

37. The method of claim 36, wherein a) the ATG5 comprises an amino acid sequence at least 95% identical to the sequence of MTDDKDVLRDVWFGRIPTCFTLYQDEITEREAEPYYLLLPRVSYLTLVTDKVKKH FQKVMRQEDISEIWFEYEGTPLKWHYPIGLLFDLLASSSALPWNITVHFKSFPEKDL LHCPSKDAIEAHFMSCMKEADALKHKSQVINEMQKKDHKQLWMGLQNDRFDQF WAINRKLMEYPAEENGFRYIPFRIYQTTTERPFIQKLFRPVAADGQLHTLGDLLKE VCPSAIDPEDGEKKNQVMIHGIEPMLETPLQWLSEHLSYPDNFLHISIIPQPTD (SEQ ID NO: 1) or
b) the ATG5 comprises an amino acid sequence at least 95% identical to the sequence of MTDDKDVLRDVWFGRIPTCFTLYQDEITEREAEPYYLLLPRVSYLTLVTDKVKKH FQKVMRQEDVSEIWFEYEGTPLKWHYPIGLLFDLLASSSALPWNITVHFKSFPEKD LLHCPSKDAVEAHFMSCMKEADALKHKSQVINEMQKKDHKQLWMGLQNDRFD QFWAINRKLMEYPPEENGFRYIPFRIYQTTTERPFIQKLFRPVAADGQLHTLGDLLR EVCPSAVAPEDGEKRSQVMIHGIEPMLETPLQWLSEHLSYPDNFLHISIVPQPTD (SEQ ID NO: 2).

38. The method of claim 35, wherein the autophagy modulator is ATG7.

39. The method of claim 38, wherein a) the ATG7 comprises an amino acid sequence at least 95% identical to the sequence of MAAATGDPGLSKLQFAPFSSALDVGFWHELTQKKLNEYRLDEAPKDIKGYYYNG DSAGLPARLTLEFSAFDMSAPTPARCCPAIGTLYNTNTLESFKTADKKLLLEQAAN EIWESIKSGTALENPVLLNKFLLLTFADLKKYHFYYWFCYPALCLPESLPLIQGPVG LDQRFSLKQIEALECAYDNLCQTEGVTALPYFLIKYDENMVLVSLLKHYSDFFQG QRTKITIGVYDPCNLAQYPGWPLRNFLVLAAHRWSSSFQSVEVVCFRDRTMQGA RDVAHSIIFEVKLPEMAFSPDCPKAVGWEKNQKGGMGPRMVNLSECMDPKRLAE SSVDLNLKLMCWRLVPTLDLDKVVSVKCLLLGAGTLGCNVARTLMGWGVRHIT FVDNAKISYSNPVRQPLYEFEDCLGGGKPKALAAADRLQKIFPGVNARGFNMSIP MPGHPVNFSSVTLEQARRDVEQLEQLIESHDVVFLLMDTRESRWLPAVIAASKRK LVINAALGFDTFVVMRHGLKKPKQQGAGDLCPNHPVASADLLGSSLFANIPGYKL GCYFCNDVVAPGDSTRDRTLDQQCTVSRPGLAVIAGALAVELMVSVLQHPEGGY AIASSSDDRMNEPPTSLGLVPHQIRGFLSRFDNVLPVSLAFDKCTACSSKVLDQYE REGFNFLAKVFNSSHSFLEDLTGLTLLHQETQAAEIWDMSDDETI (SEQ ID NO: 3) or
b) the ATG7 comprises an amino acid sequence at least 95% identical to the sequence of MGDPGLAKLQFAPFNSALDVGFWHELTQKKLNEYRLDEAPKDIKGYYYNGDSAG LPTRLTLEFSAFDMSASTPAHCCPAMGTLHNTNTLEAFKTADKKLLLEQSANEIW EAIKSGAALENPMLLNKFLLLTFADLKKYHFYYWFCCPALCLPESIPLIRGPVSLDQ RLSPKQIQALEHAYDDLCRAEGVTALPYFLFKYDDDTVLVSLLKHYSDFFQGQRT KITVGVYDPCNLAQYPGWPLRNFLVLAAHRWSGSFQSVEVLCFRDRTMQGARDV THSIIFEVKLPEMAFSPDCPKAVGWEKNQKGGMGPRMVNLSGCMDPKRLAESSV DLNLKLMCWRLVPTLDLDKVVSVKCLLLGAGTLGCNVARTLMGWGVRHVTFV DNAKISYSNPVRQPLYEFEDCLGGGKPKALAAAERLQKIFPGVNARGFNMSIPMP GHPVNFSDVTMEQARRDVEQLEQLIDNHDVIFLLMDTRESRWLPTVIAASKRKLV INAALGFDTFVVMRHGLKKPKQQGAGDLCPSHLVAPADLGSSLFANIPGYKLGCY FCNDVVAPGDSTRDRTLDQQCTVSRPGLAVIAGALAVELMVSVLQHPEGGYAIAS SSDDRMNEPPTSLGLVPHQIRGFLSRFDNVLPVSLAFDKCTACSPKVLDQYEREGF TFLAKVFNSSHSFLEDLTGLTLLHQETQAAEIWDMSDEETV (SEQ ID NO: 4).

40. The method of claim 33, wherein the vector is a lentiviral vector.


SEQUENCE LISTING (10 sequences)

SEQ ID NO: 1 - protein, 275 aa, Homo sapiens
MTDDKDVLRDVWFGRIPTCFTLYQDEITEREAEPYYLLLPRVSYLTLVTDKVKKHFQKVMRQEDISEIWFEYEGTPLKWHYPIGLLFDLLASSSALPWNITVHFKSFPEKDLLHCPSKDAIEAHFMSCMKEADALKHKSQVINEMQKKDHKQLWMGLQNDRFDQFWAINRKLMEYPAEENGFRYIPFRIYQTTTERPFIQKLFRPVAADGQLHTLGDLLKEVCPSAIDPEDGEKKNQVMIHGIEPMLETPLQWLSEHLSYPDNFLHISIIPQPTD

SEQ ID NO: 2 - protein, 275 aa, Mus sp.
MTDDKDVLRDVWFGRIPTCFTLYQDEITEREAEPYYLLLPRVSYLTLVTDKVKKHFQKVMRQEDVSEIWFEYEGTPLKWHYPIGLLFDLLASSSALPWNITVHFKSFPEKDLLHCPSKDAVEAHFMSCMKEADALKHKSQVINEMQKKDHKQLWMGLQNDRFDQFWAINRKLMEYPPEENGFRYIPFRIYQTTTERPFIQKLFRPVAADGQLHTLGDLLREVCPSAVAPEDGEKRSQVMIHGIEPMLETPLQWLSEHLSYPDNFLHISIVPQPTD

SEQ ID NO: 3 - protein, 703 aa, Homo sapiens
MAAATGDPGLSKLQFAPFSSALDVGFWHELTQKKLNEYRLDEAPKDIKGYYYNGDSAGLPARLTLEFSAFDMSAPTPARCCPAIGTLYNTNTLESFKTADKKLLLEQAANEIWESIKSGTALENPVLLNKFLLLTFADLKKYHFYYWFCYPALCLPESLPLIQGPVGLDQRFSLKQIEALECAYDNLCQTEGVTALPYFLIKYDENMVLVSLLKHYSDFFQGQRTKITIGVYDPCNLAQYPGWPLRNFLVLAAHRWSSSFQSVEVVCFRDRTMQGARDVAHSIIFEVKLPEMAFSPDCPKAVGWEKNQKGGMGPRMVNLSECMDPKRLAESSVDLNLKLMCWRLVPTLDLDKVVSVKCLLLGAGTLGCNVARTLMGWGVRHITFVDNAKISYSNPVRQPLYEFEDCLGGGKPKALAAADRLQKIFPGVNARGFNMSIPMPGHPVNFSSVTLEQARRDVEQLEQLIESHDVVFLLMDTRESRWLPAVIAASKRKLVINAALGFDTFVVMRHGLKKPKQQGAGDLCPNHPVASADLLGSSLFANIPGYKLGCYFCNDVVAPGDSTRDRTLDQQCTVSRPGLAVIAGALAVELMVSVLQHPEGGYAIASSSDDRMNEPPTSLGLVPHQIRGFLSRFDNVLPVSLAFDKCTACSSKVLDQYEREGFNFLAKVFNSSHSFLEDLTGLTLLHQETQAAEIWDMSDDETI

SEQ ID NO: 4 - protein, 698 aa, Mus sp.
MGDPGLAKLQFAPFNSALDVGFWHELTQKKLNEYRLDEAPKDIKGYYYNGDSAGLPTRLTLEFSAFDMSASTPAHCCPAMGTLHNTNTLEAFKTADKKLLLEQSANEIWEAIKSGAALENPMLLNKFLLLTFADLKKYHFYYWFCCPALCLPESIPLIRGPVSLDQRLSPKQIQALEHAYDDLCRAEGVTALPYFLFKYDDDTVLVSLLKHYSDFFQGQRTKITVGVYDPCNLAQYPGWPLRNFLVLAAHRWSGSFQSVEVLCFRDRTMQGARDVTHSIIFEVKLPEMAFSPDCPKAVGWEKNQKGGMGPRMVNLSGCMDPKRLAESSVDLNLKLMCWRLVPTLDLDKVVSVKCLLLGAGTLGCNVARTLMGWGVRHVTFVDNAKISYSNPVRQPLYEFEDCLGGGKPKALAAAERLQKIFPGVNARGFNMSIPMPGHPVNFSDVTMEQARRDVEQLEQLIDNHDVIFLLMDTRESRWLPTVIAASKRKLVINAALGFDTFVVMRHGLKKPKQQGAGDLCPSHLVAPADLGSSLFANIPGYKLGCYFCNDVVAPGDSTRDRTLDQQCTVSRPGLAVIAGALAVELMVSVLQHPEGGYAIASSSDDRMNEPPTSLGLVPHQIRGFLSRFDNVLPVSLAFDKCTACSPKVLDQYEREGFTFLAKVFNSSHSFLEDLTGLTLLHQETQAAEIWDMSDEETV

SEQ ID NO: 5 - DNA, 1000 bp, Homo sapiens
gtctggacttgtggtgcgctgccagggatccgcagcgttgccggttgtattcgctggataccagagggcggaagtgcagcagggttcagctccgacctccgcgccggtgctttttgcggctgcgcgggcttcctggagtcctgctaccgcgtccccgcaggacagtgtgtcaggcgggcagcttgccccgccgccccaccggagcgcggaatctgggcgtccccaccagtgcggggagccggaaggaggagccatagcttggagtaggtttggctttggttgaaataagaatttagcctgtatgtactgctttaactcctggaagaatgacagatgacaaagatgtgcttcgagatgtgtggtttggacgaattccaacttgtttcacgctatatcaggatgagataactgaaagggaagcagaaccatactatttgcttttgccaagagtaagttatttgacgttggtaactgacaaagtgaaaaagcactttcagaaggttatgagacaagaagacattagtgagatatggtttgaatatgaaggcacaccactgaaatggcattatccaattggtttgctatttgatcttcttgcatcaagttcagctcttccttggaacatcacagtacattttaagagttttccagaaaaagaccttctgcactgtccatctaaggatgcaattgaagctcattttatgtcatgtatgaaagaagctgatgctttaaaacataaaagtcaagtaatcaatgaaatgcagaaaaaagatcacaagcaactctggatgggattgcaaaatgacagatttgaccagttttgggccatcaatcggaaactcatggaatatcctgcagaagaaaatggatttcgttatatcccctttagaatatatcagacaacgactgaaagacctttcattcagaagctgtttcgtcctgtggctgcagatggacagttgcacacactaggagatctcctcaaagaagtttgtccttc

SEQ ID NO: 6 - DNA, 828 bp, Mus sp.
atgacagatgacaaagatgtgcttcgagatgtgtggtttggacgaattccaacttgctttactctctatcaggatgagataactgaaagagaagcagaaccatactatttgcttttgccaagagtcagctatttgacgttggtaactgacaaagtgaaaaagcactttcagaaggttatgagacaagaagatgttagtgagatatggtttgaatatgaaggcacacccctgaaatggcattatccaattggtttactatttgatcttcttgcatcaagttcagctcttccttggaacatcacagtacatttcaagagttttccagaaaaggaccttctacactgtccatccaaggatgcggttgaggctcactttatgtcgtgtatgaaagaagctgatgctttaaagcataaaagtcaagtgatcaacgaaatgcagaaaaaagaccacaagcagctctggatgggactgcagaatgacagatttgaccagttttgggccatcaaccggaaactcatggaatatcctccagaagaaaatggatttcgttatatcccctttagaatatatcagaccacgacggagcggcctttcatccagaagctgttccggcctgtggccgcagatggacagctgcacacacttggagatctcctcagagaagtctgtccttccgcagtcgcccctgaagatggagagaagaggagccaggtgatgattcacgggatagagccaatgctggaaacccctctgcagtggctgagcgagcatctgagctacccagataactttcttcatattagcattgtcccccagccaacagattga

SEQ ID NO: 7 - DNA, 1000 bp, Homo sapiens
ggaagttgagcggcggcaagaaataatggcggcagctacgggggatcctggactctctaaactgcagtttgccccttttagtagtgccttggatgttgggttttggcatgagttgacccagaagaagctgaacgagtatcggctggatgaagctcccaaggacattaagggttattactacaatggtgactctgctgggctgccagctcgcttaacattggagttcagtgcttttgacatgagtgctcccaccccagcccgttgctgcccagctattggaacactgtataacaccaacacactcgagtctttcaagactgcagataagaagctccttttggaacaagcagcaaatgagatatgggaatccataaaatcaggcactgctcttgaaaaccctgtactcctcaacaagttcctcctcttgacatttgcagatctaaagaagtaccacttctactattggttttgctatcctgccctctgtcttccagagagtttacctctcattcaggggccagtgggtttggatcaaaggttttcactaaaacagattgaagcactagagtgtgcatatgataatctttgtcaaacagaaggagtcacagctcttccttacttcttaatcaagtatgatgagaacatggtgctggtttccttgcttaaacactacagtgatttcttccaaggtcaaaggacgaagataacaattggtgtatatgatccctgtaacttagcccagtaccctggatggcctttgaggaattttttggtcctagcagcccacagatggagtagcagtttccagtctgttgaagttgtttgcttccgtgaccgtaccatgcagggggcgagagacgttgcccacagcatcatcttcgaagtgaagcttccagaaatggcatttagcccagattgtcctaaagcagttggatgggaaaagaaccagaaaggaggcatgggaccaaggatggtgaacctcagtgaatgtatggaccct

SEQ ID NO: 8 - DNA, 1000 bp, Mus sp.
atgggggaccctggactggccaagttgcagttcgccccctttaatagtgccctggacgttggcctctggcacgaactgacccagaagaagttgaacgagtaccgcctggacgaggcacccaaagacatcaagggctattactacaatggtgactctgctggtctgcccacccgcttgacgttggagttcagtgcttttgacatgagtgcctccacgcctgcccactgctgcccggccatgggaaccctgcacaacaccaacacacttgaggcttttaagacagcagacaagaagctccttctggagcagtcagcaaatgagatctgggaagccataaagtcaggtgctgctctcgaaaaccccatgctcctcaacaagtttctgctcctgaccttcgcggacctaaagaagtaccacttctactactggttttgctgccccgccctctgtcttcctgagagcatccctctaatccggggacctgtgagcttggatcaaaggctttcaccaaaacagatccaggccctggagcatgcctatgatgatctgtgtcgagccgaaggcgtcacggccctgccctacttcttattcaagtacgatgacgacactgttctggtctccttgctcaaacactacagtgatttcttccaaggtcaaaggacaaagataacagttggtgtgtacgatccctgtaacctagcccagtaccctggatggcctttgaggaattttttggtcctggcagcccacagatggagcggcagtttccagtccgttgaagtcctctgctttcgggaccgcaccatgcagggagctagagacgtgacacatagcatcatctttgaagtgaaacttccagaaatggcatttagcccagattgtcctaaagctgttggctgggagaagaaccagaaaggaggcatgggtccgaggatggtgaacctcagtggatgtatggaccccaaaaggctggctgagtcatctgtggatctgaatctca

SEQ ID NO: 9 - DNA, 22 bp, Artificial Sequence
  Description of Artificial Sequence Synthetic primer
ggccctgtaattggaatgagtc

SEQ ID NO: 10 - DNA, 20 bp, Artificial Sequence
  Description of Artificial Sequence Synthetic primer
caagatccaactacgagctt